(19) United States
(12) Patent Application Publication
(10) Pub. No.: US 2026/0258487 A1
(43) Pub. Date: Sep. 3, 2026

(54) METHODS AND COMPOSITIONS FOR MODIFICATION AND DETECTION OF 5-METHYLCYTOSINE

(71) Applicant: THE UNIVERSITY OF CHICAGO, Chicago, IL (US)

(72) Inventors: Chuan HE, Chicago, IL (US); Weixin TANG, Chicago, IL (US); Qinzhe LIU, Chicago, IL (US); Pingluan WANG, Chicago, IL (US)

(73) Assignee: THE UNIVERSITY OF CHICAGO, Chicago, IL (US)

(21) Appl. No.: 18/993,320

(22) PCT Filed: Jul. 11, 2023
(86) PCT No.: PCT/US2023/069972

§ 371 Date: Jan. 10, 2025

Related U.S. Application Data
(60) Provisional application No. 63/388,126, filed on Jul. 11, 2022.

(51) Int. Cl. C12Q 1/6869; C12Q 1/26; C12Q 1/34; C12Q 1/6806; C12Q 1/6827; C12Q 1/686

(52) U.S. Cl. CPC C12Q 1/6869; C12Q 1/26; C12Q 1/34; C12Q 1/6806; C12Q 1/6827; C12Q 1/686; G01N 2333/90245; G01N 2333/924

(57) ABSTRACT

Disclosed herein are methods and compositions for 5mC modification, detection, sequencing, and analysis. Certain aspects of the disclosure are directed to methods for modification of a 5mC in a nucleic acid molecule, including oxidation, removal, and replacement of a 5mC with a nucleobase derivative. Also disclosed are novel nucleobase derivatives, including thymine derivatives, and other compounds as well as methods for use of such derivatives and compounds in 5mC modification and analysis.

### CROSS-REFERENCE TO RELATED APPLICATIONS

This application claims priority to and the benefit of U.S. Provisional Application No. 63/388,126, filed Jul. 11, 2022, the contents of which are incorporated into the present application by reference in its entirety.

### STATEMENT OF GOVERNMENT SUPPORT

This invention was made with government support under grant number HG006827 awarded by the National Institutes of Health. The government has certain rights in the invention.

### BACKGROUND

### I. Field of the Invention

Aspects of this invention relate to at least the fields of molecular biology, biochemistry, and chemistry. Certain aspects relate to methods and compositions for modification, detection, and analysis of methylated nucleic acids.

### II. Background

Bisulfite sequencing (BS-seq) has been considered the gold standard for DNA cytosine methylation (5mC) sequencing in DNA for decades. However, conventional BS-seq suffers several major drawbacks, limiting its application in 5mC sequencing in DNA. DNA degradation caused by bisulfite treatment limits the amount of materials required for BS-seq. In addition, the reduced complexity due to C-to-U conversion poses challenges for sequence alignment as well as mutation detection in the same assay.

There exists a need for methods and compositions useful in analysis and sequencing of DNA methylation at single-base resolution, without the need for harsh bisulfite treatment.

### SUMMARY

Aspects of the present disclosure are based, at least in part, on the development of a new, bisulfite-free method for DNA methylation analysis. Also disclosed are novel nucleobase derivatives, including thymine derivatives such as N₃-thymine, as well as methods of use thereof in nucleic acid modification and in detection and analysis of DNA methylation. Accordingly, described herein, in some aspects, is a method for modifying a 5-methylcytosine (5mC) in a nucleic acid molecule, the method comprising (a) incubating the nucleic acid molecule with an agent under conditions sufficient to oxidize the 5mC to 5-carboxylcytosine (5caC) or 5-formylcytosine (5fC); (b) incubating the nucleic acid molecule with a thymine DNA glycosylase (TDG) enzyme to excise the 5caC or 5fC creating an abasic site; and (c) incubating the nucleic acid molecule with a nucleobase derivative to attach the nucleobase derivative to the nucleic acid molecule at the abasic site. Also disclosed are methods for modifying an abasic site, comprising incubating the nucleic acid molecule and a nucleobase derivative under conditions sufficient to attach the nucleobase derivative to the nucleic acid molecule at the abasic site. Further disclosed are nucleobase derivative compounds, including a compound having formula (I):

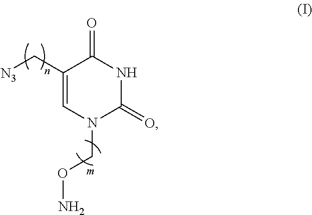

wherein n is an integer from 0 to 5 and m is an integer from 1 to 5. In some aspects, it is specifically contemplated that n is not 0, 1, 2, 3, 4, or 5 and/or m is not 1, 2, 3, 4, 5. Also disclosed is a compound having formula (II):

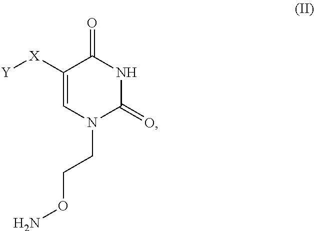

wherein X is a linker and Y is a click chemistry compatible reactive group selected from alkynes, azides, strained alkynes, dienes, dieneophiles, alkoxyamines, carbonyls, phosphines, hydrazides, thiols, alkenes, tetrazines, tetrazoles, isocyanates, isothiocyanates, and 1,3-nitrones. It is also specifically contemplated that, in certain aspects, Y is not an alkyne, azide, strained alkyne, diene, dieneophile, alkoxyamine, carbonyl, phosphine, hydrazide, thiol, alkene, tetrazine, tetrazole, isocyanate, isothiocyanate, or 1,3-nitrone. Further disclosed is a compound of formula (III):

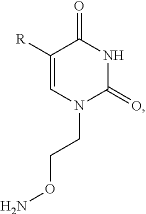

wherein R is H or alkyl. It is also specifically contemplated that, in certain aspects, R is not H. It is also specifically contemplated that, in certain aspects, R is not alkyl.

Embodiments of the present disclosure include methods for modifying a 5mC, methods for modifying a 5hmC, methods for 5mC detection, methods for modifying a nucleic acid comprising an abasic site, methods for generating an abasic site at a 5mC site, methods for synthesizing a nucleobase derivative (e.g., thymine derivative, adenine derivative, cytosine derivative, guanine derivative, uracil derivative, hypoxanthine derivative, xanthine derivative), methods for attaching a nucleobase derivative to an abasic site, methods for detection of methylated DNA, methods for methylation-specific DNA sequencing, thymine derivatives, adenine derivatives, cytosine derivatives, guanine derivatives, uracil derivatives, hypoxanthine derivatives, xanthine derivatives, and other nucleobase derivatives.

Methods of the disclosure can include at least 1, 2, 3, or more of the following steps: incubating a nucleic acid molecule with a ten-eleven translocation (TET) enzyme, incubating a nucleic acid molecule with a thymine DNA glycosylase (TDG) enzyme, incubating a nucleic acid molecule with a nucleobase derivative (e.g., a thymine derivative such as N₃-thymine), incubating a nucleic acid molecule with a beta-glucosyltransferase (BGT) enzyme, subjecting a nucleic acid molecule comprising a nucleobase derivative to a click chemistry reaction, incubating a nucleic acid molecule comprising a nucleobase derivative with a label comprising an alkyne moiety, isolating a nucleic acid molecule comprising a nucleobase derivative, isolating a plurality of nucleic acid molecules, purifying a nucleic acid molecule, sequencing a nucleic acid molecule comprising a nucleobase derivative, sequencing a plurality of nucleic acid molecules comprising nucleobase derivatives, synthesizing a thymine derivative, and synthesizing a nucleobase derivative. Any one or more of the preceding steps may be excluded from certain aspects of the disclosure. In some aspects, a method of the disclosure does not comprise bisulfite treatment. In some aspects, a method of the disclosure does not comprise incubation with bisulfite (e.g., sodium bisulfite, ammonium bisulfite, or other bisulfite source).

Disclosed herein, in some aspects, is a method for modifying a 5-methylcytosine (5mC) in a nucleic acid molecule, the method comprising (a) incubating the nucleic acid molecule with an agent under conditions sufficient to oxidize the 5mC to 5-carboxylcytosine (5caC) or 5-formylcytosine (5fC), (b) incubating the nucleic acid molecule with a thymine DNA glycosylase (TDG) enzyme to excise the 5caC or 5fC creating an abasic site, and (c) incubating the nucleic acid molecule with a nucleobase derivative to attach the nucleobase derivative to the nucleic acid molecule at the abasic site. In some aspects, the agent is a ten-eleven translocation (TET) enzyme.

Also disclosed herein, in some aspects, is a method for 5-methylcytosine (5mC) detection, the method comprising: (a) incubating a nucleic acid molecule comprising a 5mC with a TET enzyme to oxidize 5mC to 5caC or 5fC; (b) incubating the nucleic acid molecule with a TDG enzyme to excise the 5caC or 5fC and generate an abasic site; (c) incubating the nucleic acid molecule with a thymine derivative comprising an azide moiety to attach the thymine derivative to the abasic site; and (d) sequencing the nucleic acid molecule.

In some aspects, the TET enzyme is a mammalian TET enzyme. In some aspects, the TET enzyme is a murine TET enzyme. In some aspects, the TET enzyme is TET1, TET2, or TET3. In certain aspects, the TET enzyme is TET1. In some aspects, the TET enzyme is TET2. In some aspects, the TET enzyme is TET3. In some aspects, the TDG enzyme is a mammalian TDG enzyme (e.g., human TDG). In some aspects, the TDG enzyme is a murine TDG enzyme. It is also specifically contemplated that, in some aspects, the TET enzyme is not any of the specific TET enzymes disclosed herein.

In some aspects, (a), (b), and/or (c) is performed at a temperature of at least, at most, exactly, or about 25, 26, 27, 28, 29, 30, 31, 32, 33, 34, 35, 36, 37, 38, 39, 40, 41, 42, 43, 44, or 45° C., including any range or value derivable therein. In certain aspects, it is specifically contemplated that (a), (b), and/or (c) is not performed at 25, 26, 27, 28, 29, 30, 31, 32, 33, 34, 35, 36, 37, 38, 39, 40, 41, 42, 43, 44, or 45° C. In some aspects, (c) is performed at between 3° and 40° C. In some aspects, (c) is performed at about or exactly 37° C. In some aspects, (a), (b), and/or (c) is performed for less than, about, or exactly 6, 5, 4, 3, 2, or 1 hours, including any range or value derivable therein. In some aspects, (c) is performed for less than or equal to 4 hours. In some aspects, (a), (b), and/or (c) is performed at a pH of at least, at most, exactly, or about 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7, including any range or value derivable therein. In some aspects, (c) is performed at a pH from 5.5 to 6.5. It is specifically contemplated that, in certain aspects, (a), (b), and/or (c) is not performed at a pH of at least, at most, exactly, or about 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7, including any range or value derivable therein.

Also disclosed herein, in some aspects, is a method for modifying a nucleic acid molecule comprising an abasic site, the method comprising incubating the nucleic acid molecule and a nucleobase derivative under conditions sufficient to attach the nucleobase derivative to the nucleic acid molecule at the abasic site. In some aspects, the method is performed at a temperature of at least, at most, exactly, or about 25, 26, 27, 28, 29, 30, 31, 32, 33, 34, 35, 36, 37, 38, 39, 40, 41, 42, 43, 44, or 45° C., including any range or value derivable therein. In some aspects, the method is performed at between 3° and 40° C. In some aspects, the method is performed at about or exactly 37° C. In some aspects, the method is performed for less than, about, or exactly 6, 5, 4, 3, 2, or 1 hours, including any range or value derivable therein. In some aspects, the method is performed for less than or equal to 4 hours. In some aspects, the method is performed at a pH of at least, at most, exactly, or about 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7, including any range or value derivable therein. In some aspects, the method is performed at a pH from 5.5 to 6.5.

In some aspects, the nucleobase derivative comprises an azide moiety. In some aspects, the nucleobase derivative is a thymine derivative. In some aspects, the thymine derivative is a compound of formula I:

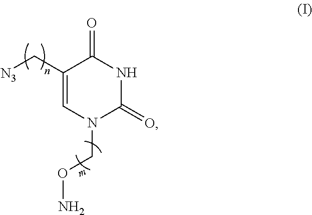

wherein n is an integer from 0 to 5 and m is an integer from 1 to 5. In some aspects, n is 1, 2, 3, 4, or 5. In some aspects, n is 3. In some aspects, m is 1, 2, 3, 4, or 5. In some aspects, m is 2. In some aspects, it is specifically contemplated that n is not 0, 1, 2, 3, 4, or 5 and/or m is not 1, 2, 3, 4, 5. In some aspects, the thymine derivative is N₃-T:

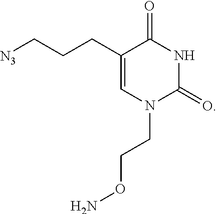 [SMILES: [N-]=[N+]=NCCCc1cn(CCON)c(=O)[nH]c1=O]

In some aspects, the nucleobase derivative is an adenine derivative.

In some aspects, the method further comprises sequencing the nucleic acid molecule. In some aspects, the nucleic acid molecule is a deoxyribonucleic acid (DNA) molecule. The DNA molecule may be, for example, a genomic DNA molecule, a tumor DNA molecule, a fetal DNA molecule, a cell-free DNA (cfDNA) molecule, or any other DNA molecule. In some aspects, the nucleic acid molecule was obtained from a sample comprising at or below 200, 150, 100, 50, or 25 ng total nucleic acid, or less. In some aspects, the nucleic acid molecule was obtained from a sample comprising at or below 200, 150, 100, 50, or 25 cells, or less.

In some aspects, the method further comprises subjecting the nucleic acid molecule to a click chemistry reaction to attach a label to the nucleic acid molecule. In some aspects, the label comprises an alkyne moiety. In some aspects, the label is a dibenzocyclooctyne-modified biotin (DBCO-biotin). In some aspects, the method further comprises incubating the nucleic acid molecule with streptavidin. In some aspects, the method further comprises subjecting the nucleic acid molecule to a polymerase chain reaction.

In some aspects, the nucleic acid molecule comprises a 5-hydroxymethylcytosine (5hmC), and the method further comprises incubating the nucleic acid molecule with a beta-glucosyltransferase (BGT) enzyme to glycosylate the 5hmC to 5-glyceryl-methylcytosine (5gmC) prior to (a). In some aspects, the BGT enzyme is a mammalian BGT enzyme (e.g., human βGT). In some aspects, the method does not comprise bisulfite treatment.

Also disclosed herein, in some aspects, is a compound of formula (I):

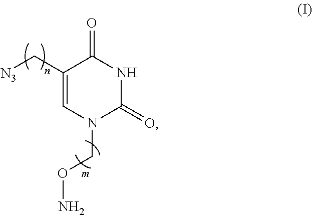

wherein n is an integer from 0 to 5 and m is an integer from 1 to 5. In some aspects, n is 0, 1, 2, 3, 4, or 5. In some aspects, m is 1, 2, 3, 4, or 5. In some aspects, n is 0 and m is 1, 2, 3, 4, or 5. In some aspects, n is 1 and m is 1, 2, 3, 4, or 5. In some aspects, n is 2 and m is 1, 2, 3, 4, or 5. In some aspects, n is 3 and m is 1, 2, 3, 4, or 5. In some aspects, n is 4 and m is 1, 2, 3, 4, or 5. In some aspects, n is 5 and m is 1, 2, 3, 4, or 5. In some aspects, n is 3 and m is 2. In some aspects, it is specifically contemplated that n is not 0, 1, 2, 3, 4, or 5 and m is not 1, 2, 3, 4, 5. In some aspects, the compound is further defined as:

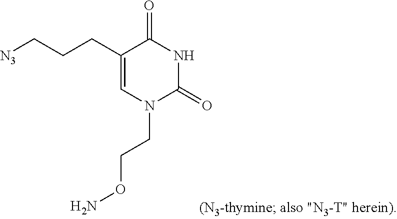 [SMILES: [N-]=[N+]=NCCCc1cn(CCON)c(=O)[nH]c1=O]

Also disclosed herein, in some aspects, is a compound having formula (II):

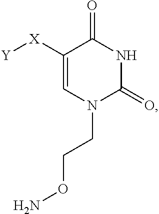

wherein X is a linker and Y is a click chemistry compatible reactive group selected from alkynes, azides, strained alkynes, dienes, dieneophiles, alkoxyamines, carbonyls, phosphines, hydrazides, thiols, alkenes, tetrazines, tetrazoles, isocyanates, isothiocyanates, and 1,3-nitrones. It is also specifically contemplated that, in certain aspects, Y is not an alkyne, azide, strained alkyne, diene, dieneophile, alkoxyamine, carbonyl, phosphine, hydrazide, thiol, alkene, tetrazine, tetrazole, isocyanate, isothiocyanate, or 1,3-nitrone. X may be any suitable linker known in the art. In some aspects, X is a C1 to C10 alkyl linker. In some aspects, X is CH₂. In some aspects, X is CH₂CH₂. In some aspects, X is CH₂CH₂CH₂. In some aspects, X is a polyethylene glycol linker. In some aspects, X is an amide linker. In some aspects, X is an alkyl and aryl mixture linker. In some aspects, X is an alkyl and heterocycle mixture linker. In some aspects, X is not a C1 to C10 alkyl linker. In some aspects, X is not CH₂. In some aspects, X is not CH₂CH₂. In some aspects, X is not CH₂CH₂CH₂. In some aspects, X is not a polyethylene glycol linker. In some aspects, X is not an amide linker. In some aspects, X is not an alkyl and aryl mixture linker. In some aspects, X is not an alkyl and heterocycle mixture linker. In some aspects, Y is azide.

Further disclosed herein, in some aspects, is a compound having formula (III):

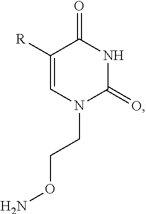

wherein R is H or alkyl. In some aspects, R is H. In some aspects, R is methyl, ethyl, n-propyl, isopropyl, n-butyl, iso-butyl, or sec-butyl. In certain aspects, R is methyl. In some aspects, it is specifically contemplated that R is not methyl, ethyl, n-propyl, isopropyl, n-butyl, iso-butyl, or sec-butyl. In some aspects, the compound is further defined as:

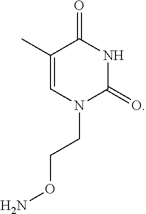

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of”′ any of the ingredients or steps disclosed limits the scope of the claim to the specified materials or steps which do not materially affect the basic and novel characteristic of the claimed invention.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. FIG. 1A shows a schematic diagram of TT-5mC-seq. 5mCs in genomic DNA are converted to 5caCs by TET-mediated oxidation. After TDG excision, abasic site (AP-site) is created at the original 5mC. N₃-T can specifically react with AP-site and leads to a 5mC-to-T mutation can be used to identify 5mC sites genome-wide at single-base substitution with or without enrichment. FIG. 1B shows another schematic diagram of TT-5mC-seq including a step of modification of 5hmC via β-glucosyltransferase labeling FIG. 1C shows the structure of N₃-T and Biotin-N₃-T modified DNA. FIG. 1D shows the structure of 10-mer double strand model DNA with 5mC modification on both sides.

FIGS. 2A-2B. MALDI-TOF MS characterization of 5mC, 5caC, AP site, and N₃-T containing 10-mer DNA in a model experiment. FIG. 2A shows MALDI-TOF of 5mC, 5caC, AP site, and N₃-T containing 10-mer DNA, respectively, with the calculated molecular weight and observed molecular weight indicated. FIG. 2B shows corresponding reactions of the mTET oxidation, TDG base excision and the subsequent reaction with N₃-T. Reactions were performed in duplex DNA with the complementary strand.

FIGS. 3A-3B. FIG. 3A shows Sanger sequencing results for a model DNA containing fully methylated CpG sites before (top) and after (bottom) TT-5mC-seq. 5mC is converted to T after TT-5mC-seq. FIG. 3B shows results from a dot blot assay of TT-5mC-seq. Dot 1: Model DNA oligo labeled with N₃-T and then further labeled with DBCO—S-S-PEG3-biotin.; dot 2: Model DNA oligo with no treatment.

FIGS. 4A-4B. NGS result on 164mer spike in suggests improved sequencing quality of TT-5mC over TAPS. FIG. 4A shows undesired C-to-DHU conversion rate; TT-5mC reduced the background noise by 66% compared to TAPS. FIG. 4B shows results demonstrating that TT-5mC gave comparable mutation rate on all four 5mC sites.

FIG. 5 shows the scheme for synthesis of N₃-T.

FIGS. 6A and 6B. Characterization of alternative base substitutions. FIG. 6A shows MALDI-TOF analysis of thymine derivative- and adenine derivative-containing 10-mer DNA, with the calculated molecular weight and observed molecular weight indicated. FIG. 6B shows NGS results on 164mer spike-in probe treated with alternative base substitutions.

FIG. 7 shows MALDI-TOF MS analysis of reaction between oligo with abasic site and N₃-T in different concentrations.

### DETAILED DESCRIPTION OF THE INVENTION

DNA cytosine methylation (5mC) has been widely studied and characterized. 5mC is involved in a wide range of biological processes in mammalian cells. It is deposited by DNA methyltransferases (DNMT) and constitutes ~2-6% of the total cytosines in human genomic DNA. Currently, bisulfite sequencing is considered the “gold standard” for DNA methylation analysis. However, bisulfite sequencing suffers from various drawbacks, including DNA degradation due to harsh treatment conditions, making it less suited for low input DNA such as cell-free DNA (cfDNA) samples.

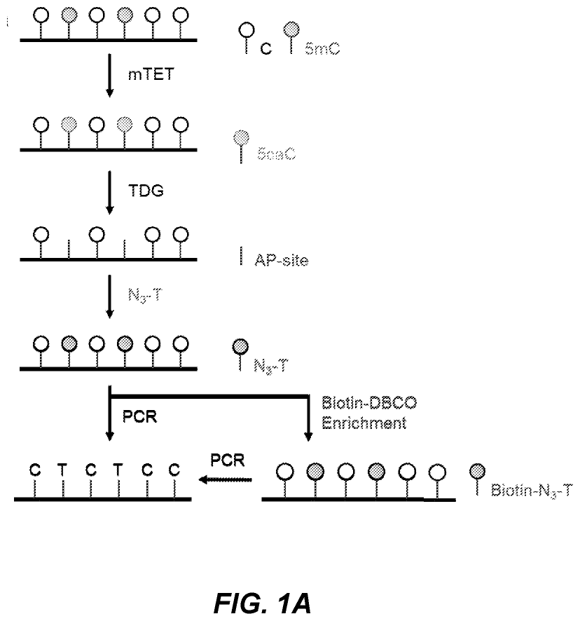

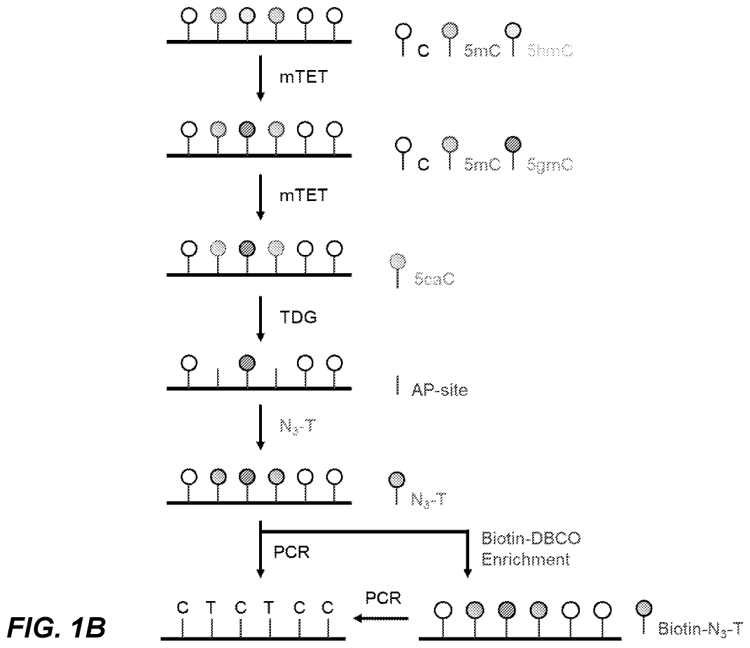

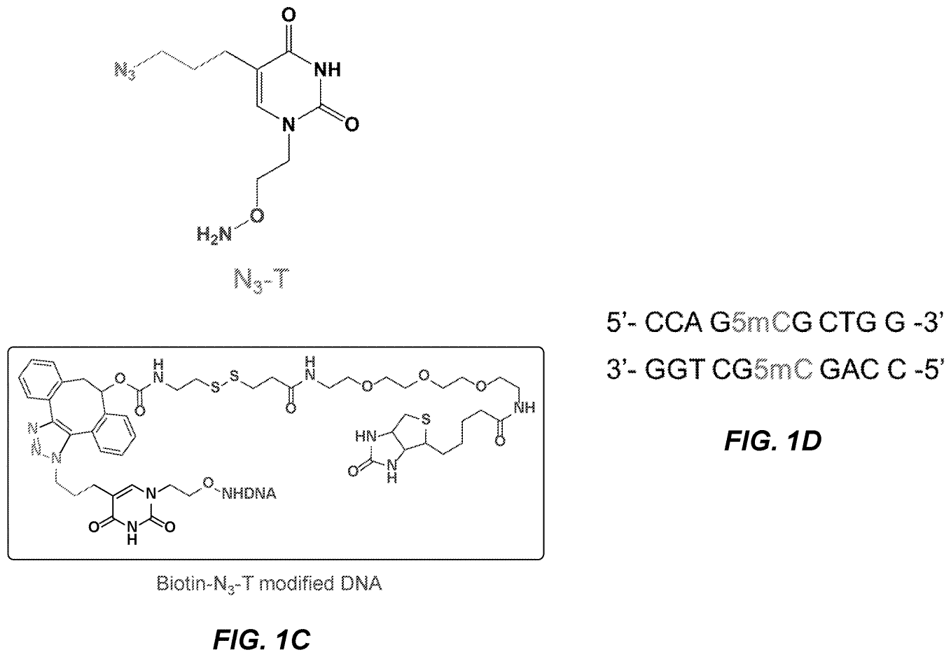

Described herein are methods and compositions which serve to overcome these and other challenges. Aspects of the disclosure are directed to methods which achieve base-resolution 5mC sequencing without the use of toxic chemicals. The disclosed methods further enable labeling, isolation, and/or enrichment of 5mC-containing DNA fragments, which can increase signal and reduce costs. An example of an embodiment method of the disclosure is shown in FIG. 1A. Further examples are described elsewhere herein.

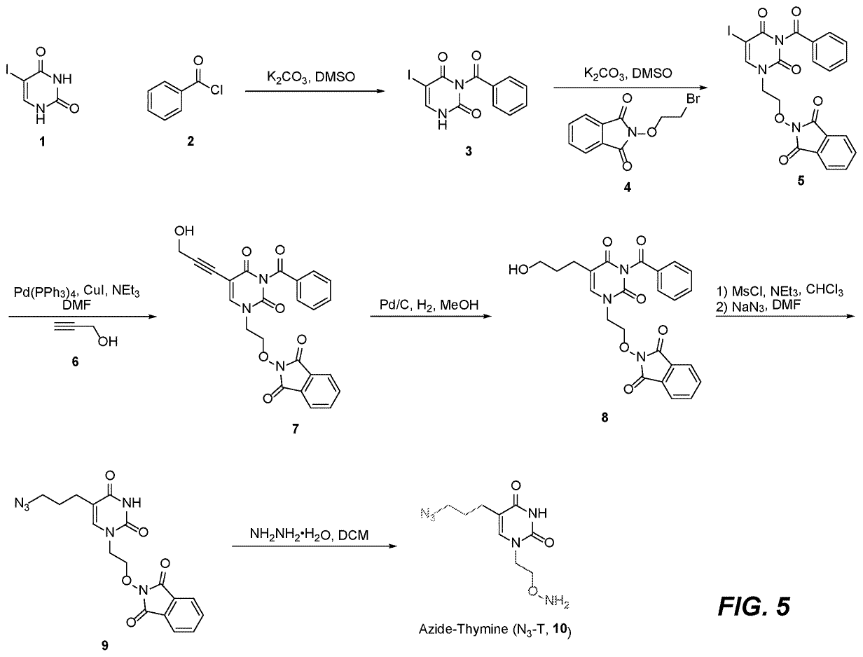

Also disclosed are novel compounds, including nucleobase derivatives, and methods for use of such compounds in 5mC modification and analysis. Example embodiments of novel compounds of the disclosure are shown in FIG. 1B and FIG. 5, and described elsewhere herein.

I. 5-methylcytosine Modification and Analysis

Aspects of the present disclosure are directed to methods for modification and analysis of 5-methylcytosine (5mC) in nucleic acid (e.g., DNA). Certain aspects further include methods for modifying a nucleic acid molecule comprising an abasic site. In some aspects, an abasic site of a nucleic acid molecule is at a position previously occupied by a 5mC.

In some aspects, methods of the disclosure comprise incubating a nucleic acid comprising a 5mC with an agent under conditions sufficient to oxidize the 5mC to 5-carboxylcytosine (5caC) or 5-formylcytosine (5fC). In some aspects, the 5mC is oxidized to 5caC. In some aspects, the 5mC is oxidized to 5fC. An agent may be any oxidizing agent capable of oxidizing 5mC to 5caC or f5C, including chemical and biological agents. In some aspects, an agent capable of oxidizing 5mC to 5caC or f5C is a ten-eleven translocation (TET) enzyme. As used herein, a TET enzyme (also “methylcytosine dioxygenase”) describes an enzyme having methylcytosine dioxygenase activity, characterized by Enzyme Commission (EC) number 1.14.11.n2. TET enzymes include human, murine, and other mammalian TET enzymes. Example TET enzymes contemplated herein include human TET1 (UniProtKB/Swiss-Prot accession number Q8NFU7), human TET2 (UniProtKB/Swiss-Prot accession number Q6N021), human TET3 (UniProtKB/Swiss-Prot accession number O43151), murine TET1 (UniProtKB/Swiss-Prot accession number Q3URK3), murine TET2 (UniProtKB/Swiss-Prot accession number Q4JK59), and murine TET3 (UniProtKB/Swiss-Prot accession number Q8BG87). In some aspects, a TET enzyme used herein is murine TET1.

In some aspects, a TET enzyme used herein is human TET1. In some aspects, a TET enzyme used herein is murine TET2. In some aspects, a TET enzyme used herein is human TET2. Conditions sufficient to oxidize a 5mC to 5caC or 5fC include, for example, sufficient temperature, time, buffer, pH, and/or other conditions which enable oxidation of 5mC to 5caC or 5fC.

In certain aspects, methods of the present disclosure comprise incubating a nucleic acid molecule comprising a 5caC or 5fC with a thymine DNA glycosylase (TDG) enzyme to excise the 5caC or 5fC. As used herein, a “TDG enzyme” describes an enzyme having thymine DNA glycosylate activity, characterized by Enzyme Commission (EC) number 3.2.2.29. TDG enzymes include human, murine, and other mammalian TDG enzymes. Example TDG enzymes contemplated herein include human TDG (UniProtKB/Swiss-Prot accession number Q13569) and murine TDG (UniProtKB/Swiss-Prot accession number P56581). In some aspects, a TDG enzyme used herein is human TDG. In some aspects, a TDG enzyme used herein is murine TDG. Conditions sufficient to excise a 5caC or 5fC with a TDG enzyme include, for example, sufficient temperature, time, buffer, pH, and/or other conditions which enable excision of a 5caC or 5fC.

In some aspects, methods of the present disclosure comprise incubating a nucleic acid molecule comprising an abasic site with a nucleobase derivative to attach the nucleobase derivative to the nucleic acid molecule at the abasic site. A nucleobase derivative may be any nucleobase derivative disclosed herein. In certain aspects, the nucleobase derivative comprises an azide moiety. The nucleobase derivative may be a thymine derivative, adenine derivative, cytosine derivative, guanine derivative, uracil derivative, hypoxanthine derivative, xanthine derivative, or other purine or pyrimidine derivative. For example, the nucleobase derivative may be a compound of formula (I), (II), or (III) as described herein. In one aspect the nucleobase derivative is N₃-thymine. Conditions sufficient to attach the nucleobase derivative to the nucleic acid at the abasic site include, for example, sufficient temperature, time, buffer, pH, and/or other conditions which enable attachment of the nucleobase derivative to the nucleic acid at the abasic site. In some aspects, the incubating is performed for at least, at most, about, or exactly 6, 5, 4, 3, 2, or 1 hours, including any range or value derivable therein. In some aspects, the incubating is performed at a pH of at least, at most, exactly, or about 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7, including any range or value derivable therein (e.g., 5.5-6.5). In some aspects, the incubating is performed at a temperature of at least, at most, exactly, or about 25, 26, 27, 28, 29, 30, 31, 32, 33, 34, 35, 36, 37, 38, 39, 40, 41, 42, 43, 44, or 45° C. The skilled artisan will recognize that the conditions sufficient for attachment of a nucleobase derivative to a nucleic acid at an abasic site may be modified based on evaluation of efficacy of nucleobase attachment, for example via monitoring by MALDI-TOF mass spectrometry or other suitable analytical technique.

As disclosed herein, certain advantages of replacing a 5mC in a nucleic acid with a nucleobase derivative include the ability to detect, manipulate, and/or enrich for nucleic acids comprising the nucleobase derivative prior to further processing (e.g., PCR, sequencing, etc.). Accordingly, in some aspects, methods of the disclosure comprise attaching a label to a nucleobase derivative (e.g., a nucleobase derivative attached to a nucleic acid). A label may be any molecule useful in detection, manipulation, and/or enrichment of a nucleic acid. For example, in some cases, a label is an affinity tag. Affinity tags contemplated herein include, for example, biotin and derivatives thereof, streptavidin and derivatives thereof, and polypeptide tags (e.g., polyhistidine tags). Additional affinity tags are recognized in the art and contemplated herein. Accordingly, in some aspects, methods of the disclosure comprise subjecting a nucleic acid molecule comprising a nucleobase derivative (e.g., a nucleobase derivative comprising an azide moiety) to a click chemistry reaction to attach an affinity tag to the nucleic acid molecule. In some aspects, the affinity tag is a dibenzocyclooctyne-modified biotin (DBCO-biotin). The method may further comprise contacting the nucleic acid molecule comprising the affinity tag with a molecule having affinity for the affinity tag. For example, where the affinity tag is biotin (or a biotin derivative), the method may further comprise contacting the nucleic acid molecule with streptavidin. Additional click chemistry compatible affinity tags are recognized in the art and contemplated herein. In some cases, a label is a fluorescent label, a radiolabel, or other detectable label. Various detectable labels are recognized in the art and contemplated herein.

In certain cases, a method of disclosure includes incubating a nucleic acid molecule comprising a 5-hydroxymethylcytosine (5hmC) with a beta-glucosyltransferase (βGT) enzyme to glycosylate the 5hmC to 5-glyceryl-methylcytosine (5gmC). In other cases, a method of the disclosure does not include incubating a nucleic acid molecule comprising a 5hmC with a βGT enzyme. In some aspects, a method of the disclosure does not include bisulfite treatment.

A method of the disclosure may comprise, in certain cases, subjecting a nucleic acid molecule comprising a nucleobase derivative to a polymerase chain reaction. In some aspects, a method of the disclosure comprises sequencing a nucleic acid molecule comprising a nucleobase derivative. For example, a method may comprise sequencing a nucleic acid molecule comprising a thymine derivative to determine the location of the 5mC in the original nucleic acid molecule.

### II. Nucleobase Derivatives

Aspects of the present disclosure are directed to nucleobase derivatives. As used herein, a “nucleobase derivative” describes a molecule or compound capable of being read as a nucleobase in a sequencing or other nucleic acid analysis reaction, but having a modified structure compared to a natural nucleobase. A “nucleobase,” (also “nucleoside base,” “nitrogenous base,” or “base”) as used herein, is a term widely recognized in the art, and describes a purine or pyrimidine molecule or derivative thereof, for example a thymine (T), adenine (A), cytosine (C), guanine (G), hypoxanthine (I), xanthine (X), or uracil (U) molecule. The term “nucleobase” also describes a region of a molecule (e.g., a nucleoside, nucleotide, or nucleic acid molecule) comprising a purine or pyrimidine (e.g., T, A, C, G, I, X, or U).

Example nucleobase derivatives include thymine derivatives, adenine derivatives, cytosine derivatives, guanine derivatives, uracil derivatives, hypoxanthine derivatives, xanthine derivatives, and other purine or pyrimidine derivatives. In some aspects, disclosed herein are novel thymine derivatives. In certain embodiments, disclosed is a thymine derivative having formula (I)

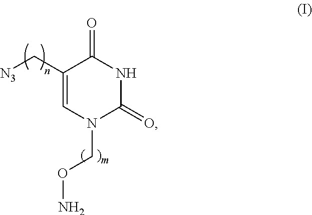

wherein n is an integer from 0 to 5 and m is an integer from 1 to 5. In some aspects, n is 0, 1, 2, 3, 4, or 5. In some aspects, n is 3. In some aspects, m is 1, 2, 3, 4, or 5. In some aspects, m is 2. In some aspects, n is 0 and m is 1, 2, 3, 4, or 5. In some aspects, n is 1 and m is 1, 2, 3, 4, or 5. In some aspects, n is 2 and m is 1, 2, 3, 4, or 5. In some aspects, n is 3 and m is 1, 2, 3, 4, or 5. In some aspects, n is 4 and m is 1, 2, 3, 4, or 5. In some aspects, n is 5 and m is 1, 2, 3, 4, or 5. In some aspects, n is 2 and m is 2.

In certain aspects, disclosed is a thymine derivative having formula:

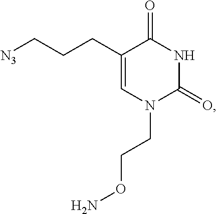 [SMILES: [N-]=[N+]=NCCCc1cn(CCON)c(=O)[nH]c1=O]

described herein as “N₃-thymine” and “N₃-T”.

Also disclosed is a thymine derivative having formula (II):

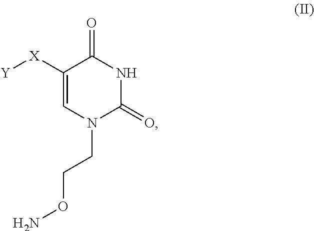

wherein X is a linker and Y is a click chemistry compatible reactive group selected from alkynes, azides, strained alkynes, dienes, dieneophiles, alkoxyamines, carbonyls, phosphines, hydrazides, thiols, alkenes, tetrazines, tetrazoles, isocyanates, isothiocyanates, 1,3-nitrones, and other click chemistry compatible reactive groups recognized in the art. The linker (X) can be, for example, a C1, C2, C3, C4, C5, C6, C7, C8, C9, or C10 alkyl, or polyethylene glycol linker (one or more embodiments can be specifically excluded). In some aspects, X is a amide linker. In some aspects, X is an alkyl and aryl mixture linker. In some aspects, X is an alkyl and heterocycle mixture linker. In certain aspects, X is CH₂. In other aspects, X is CH₂CH₂. In further aspects, X is CH₂CH₂CH₂. In some aspects, Y is azide (N₃).

Further disclosed, in some aspects, is a thymine derivative that does not comprise a click chemistry compatible reactive group. For example, in some aspects, disclosed is a compound having formula (III):

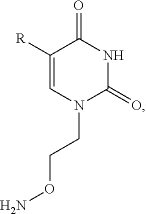

wherein R is H or alkyl. In some aspects, R is methyl, ethyl, n-propyl, isopropyl, n-butyl, iso-butyl, or sec-butyl. In certain aspects, R is methyl. Accordingly, aspects of the disclosure are directed to a compound having formula:

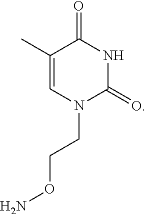

In some aspects, a nucleobase derivative of the disclosure is an adenine derivative. In some aspects, an adenine derivative of the disclosure is a compound having formula:

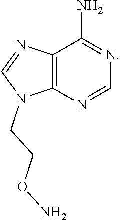 [SMILES: NOCCn1cnc2c(N)ncnc21]

In some aspects, an adenine derivative of the disclosure is a compound having formula:

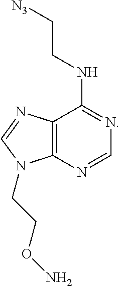 [SMILES: [N-]=[N+]=NCCNc1ncnc2c1ncn2CCON]

In some aspects, a nucleobase derivative of the disclosure is a cytosine derivative. In some aspects, a cytosine derivative of the disclosure is a compound having formula:

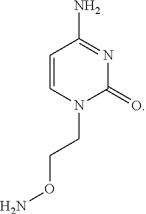 [SMILES: NOCCn1ccc(N)nc1=O]

In some aspects, a cytosine derivative of the disclosure is a compound having formula:

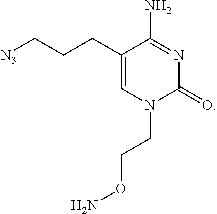 [SMILES: [N-]=[N+]=NCCCc1cn(CCON)c(=O)nc1N]

In some aspects, a nucleobase derivative of the disclosure is a guanine derivative. In some aspects, a guanine derivative of the disclosure is a compound having formula:

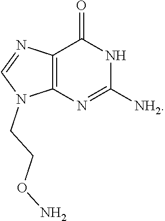 [SMILES: NOCCn1cnc2c(=O)[nH]c(N)nc21]

In some aspects, a guanine derivative of the disclosure is a compound having formula:

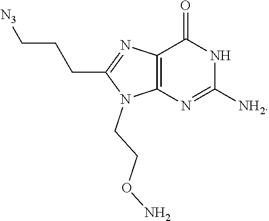 [SMILES: [N-]=[N+]=NCCCc1nc2c(=O)[nH]c(N)nc2n1CCON]

In some aspects, a nucleobase derivative of the disclosure is a uracil derivative. In some aspects, a uracil derivative of the disclosure is a compound having formula:

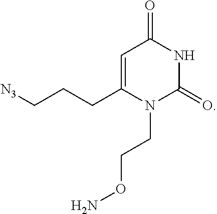 [SMILES: [N-]=[N+]=NCCCc1cc(=O)[nH]c(=O)n1CCON]

In some aspects, a uracil derivative of the disclosure is a compound having formula:

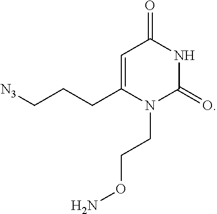 [SMILES: [N-]=[N+]=NCCCc1cc(=O)[nH]c(=O)n1CCON]

In some aspects, a nucleobase derivative of the disclosure is a hypoxanthine derivative. In some aspects, a hypoxanthine derivative of the disclosure is a compound having formula:

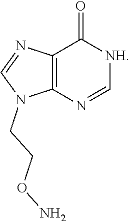 [SMILES: NOCCn1cnc2c(=O)[nH]cnc21]

In some aspects, a hypoxanthine derivative of the disclosure is a compound having formula:

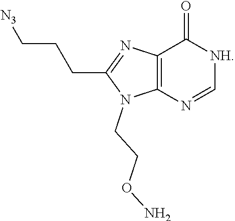 [SMILES: [N-]=[N+]=NCCCc1nc2c(=O)[nH]cnc2n1CCON]

In some aspects, a nucleobase derivative of the disclosure is a xanthine derivative. In some aspects, a xanthine derivative of the disclosure is a compound having formula:

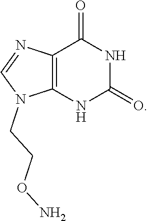 [SMILES: NOCCn1cnc2c(=O)[nH]c(=O)[nH]c21]

In some aspects, a xanthine derivative of the disclosure is a compound having formula:

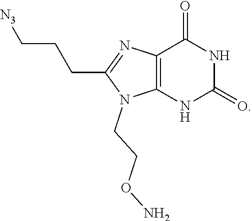 [SMILES: [N-]=[N+]=NCCCc1nc2c(=O)[nH]c(=O)[nH]c2n1CCON]

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms (C1-20 aliphatic). In certain embodiments, the aliphatic group has 1-10 carbon atoms (C1-10 aliphatic). In certain embodiments, the aliphatic group has 1-6 carbon atoms (C1-6 aliphatic). In certain embodiments, the aliphatic group has 1-5 carbon atoms (C1-5 aliphatic). In certain embodiments, the aliphatic group has 1~4 carbon atoms (C1-4 aliphatic). In certain embodiments, the aliphatic group has 1-3 carbon atoms (C1-3 aliphatic). In certain embodiments, the aliphatic group has 1-2 carbon atoms (C1-2 aliphatic). Aliphatic group substituents include, but are not limited to, any of the substituents described herein, that result in the formation of a stable moiety.

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed herein contains 1-20 carbon atoms (C1-20 alkyl). In another embodiment, the alkyl group employed contains 1-15 carbon atoms (C1-15 alkyl). In another embodiment, the alkyl group employed contains 1-10 carbon atoms (C1-10 alkyl). In another embodiment, the alkyl group employed contains 1-8 carbon atoms (C1-8 alkyl). In another embodiment, the alkyl group employed contains 1-6 carbon atoms (C1-6 alkyl). In another embodiment, the alkyl group employed contains 1-5 carbon atoms (C1-5 alkyl). In another embodiment, the alkyl group employed contains 1~4 carbon atoms (C1-4 alkyl). In another embodiment, the alkyl group employed contains 1-3 carbon atoms (C1-3 alkyl). In another embodiment, the alkyl group employed contains 1-2 carbon atoms (C1-2 alkyl). Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents. Alkyl group substituents include, but are not limited to, any of the substituents described herein, that result in the formation of a stable moiety. The term “alkylene,” as used herein, refers to a biradical derived from an alkyl group, as defined herein, by removal of two hydrogen atoms. Alkylene groups may be cyclic or acyclic, branched or unbranched, substituted or unsubstituted. Alkylene group substituents include, but are not limited to, any of the substituents described herein, that result in the formation of a stable moiety.

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed herein contains 2-20 carbon atoms (C2-20 alkenyl). In some embodiments, the alkenyl group employed herein contains 2-15 carbon atoms (C2-15 alkenyl). In another embodiment, the alkenyl group employed contains 2-10 carbon atoms (C2-10 alkenyl). In still other embodiments, the alkenyl group contains 2-8 carbon atoms (C2-8 alkenyl). In yet other embodiments, the alkenyl group contains 2-6 carbons (C2-6 alkenyl). In yet other embodiments, the alkenyl group contains 2-5 carbons (C2-5 alkenyl). In yet other embodiments, the alkenyl group contains 2-4 carbons (C2-4 alkenyl). In yet other embodiments, the alkenyl group contains 2-3 carbons (C2-3 alkenyl). In yet other embodiments, the alkenyl group contains 2 carbons (C2 alkenyl). Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety. The term “alkenylene,” as used herein, refers to a biradical derived from an alkenyl group, as defined herein, by removal of two hydrogen atoms. Alkenylene groups may be cyclic or acyclic, branched or unbranched, substituted or unsubstituted. Alkenylene group substituents include, but are not limited to, any of the substituents described herein, that result in the formation of a stable moiety.

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed herein contains 2-20 carbon atoms (C2-20alkynyl). In some embodiments, the alkynyl group employed herein contains 2-15 carbon atoms (C2-15alkynyl). In another embodiment, the alkynyl group employed contains 2-10 carbon atoms (C2-10alkynyl). In still other embodiments, the alkynyl group contains 2-8 carbon atoms (C2-8alkynyl). In still other embodiments, the alkynyl group contains 2-6 carbon atoms (C2-6alkynyl). In still other embodiments, the alkynyl group contains 2-5 carbon atoms (C2-5alkynyl). In still other embodiments, the alkynyl group contains 2-4 carbon atoms (C2-4alkynyl). In still other embodiments, the alkynyl group contains 2-3 carbon atoms (C2-3alkynyl). In still other embodiments, the alkynyl group contains 2 carbon atoms (C2alkynyl). Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety. The term “alkynylene,” as used herein, refers to a biradical derived from an alkynylene group, as defined herein, by removal of two hydrogen atoms. Alkynylene groups may be cyclic or acyclic, branched or unbranched, substituted or unsubstituted. Alkynylene group substituents include, but are not limited to, any of the substituents described herein, that result in the formation of a stable moiety.

The term “carbocyclic” or “carbocyclyl” as used herein, refers to an as used herein, refers to a cyclic aliphatic group containing 3-10 carbon ring atoms (C3-10carbocyclic). Carbocyclic group substituents include, but are not limited to, any of the substituents described herein, that result in the formation of a stable moiety.

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that further contains one or more heteroatoms (e.g., oxygen, sulfur, nitrogen, phosphorus, or silicon atoms) between carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl,” “heteroalkynyl,” and the like. Furthermore, as used herein, the terms “heteroalkyl,” “heteroalkenyl,” “heteroalkynyl,” and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms and 1-6 heteroatoms (C1-20 heteroaliphatic). In certain embodiments, the heteroaliphatic group contains 1-10 carbon atoms and 1-4 heteroatoms (C1-10 heteroaliphatic). In certain embodiments, the heteroaliphatic group contains 1-6 carbon atoms and 1-3 heteroatoms (C1-6 heteroaliphatic). In certain embodiments, the heteroaliphatic group contains 1-5 carbon atoms and 1-3 heteroatoms (C1-5 heteroaliphatic). In certain embodiments, the heteroaliphatic group contains 1~4 carbon atoms and 1-2 heteroatoms (C1-4 heteroaliphatic). In certain embodiments, the heteroaliphatic group contains 1-3 carbon atoms and 1 heteroatom (C1-3 heteroaliphatic). In certain embodiments, the heteroaliphatic group contains 1-2 carbon atoms and 1 heteroatom (C1-2 heteroaliphatic). Heteroaliphatic group substituents include, but are not limited to, any of the substituents described herein, that result in the formation of a stable moiety.

The term “heteroalkyl,” as used herein, refers to an alkyl moiety, as defined herein, which contain one or more heteroatoms (e.g., oxygen, sulfur, nitrogen, phosphorus, or silicon atoms) in between carbon atoms. In certain embodiments, the heteroalkyl group contains 1-20 carbon atoms and 1-6 heteroatoms (C1-20 heteroalkyl). In certain embodiments, the heteroalkyl group contains 1-10 carbon atoms and 1-4 heteroatoms (C1-10 heteroalkyl). In certain embodiments, the heteroalkyl group contains 1-6 carbon atoms and 1-3 heteroatoms (C1-6 heteroalkyl). In certain embodiments, the heteroalkyl group contains 1-5 carbon atoms and 1-3 heteroatoms (C1-5 heteroalkyl). In certain embodiments, the heteroalkyl group contains 1~4 carbon atoms and 1-2 heteroatoms (C1-4 heteroalkyl). In certain embodiments, the heteroalkyl group contains 1-3 carbon atoms and 1 heteroatom (C1-3 heteroalkyl). In certain embodiments, the heteroalkyl group contains 1-2 carbon atoms and 1 heteroatom (C1-2 heteroalkyl). The term “heteroalkylene,” as used herein, refers to a biradical derived from an heteroalkyl group, as defined herein, by removal of two hydrogen atoms. Heteroalkylene groups may be cyclic or acyclic, branched or unbranched, substituted or unsubstituted. Heteroalkylene group substituents include, but are not limited to, any of the substituents described herein, that result in the formation of a stable moiety.

The term “heteroalkenyl,” as used herein, refers to an alkenyl moiety, as defined herein, which further contains one or more heteroatoms (e.g., oxygen, sulfur, nitrogen, phosphorus, or silicon atoms) in between carbon atoms. In certain embodiments, the heteroalkenyl group contains 2-20 carbon atoms and 1-6 heteroatoms (C2-20 heteroalkenyl). In certain embodiments, the heteroalkenyl group contains 2-10 carbon atoms and 1-4 heteroatoms (C2-10 heteroalkenyl). In certain embodiments, the heteroalkenyl group contains 2-6 carbon atoms and 1-3 heteroatoms (C2-6 heteroalkenyl). In certain embodiments, the heteroalkenyl group contains 2-5 carbon atoms and 1-3 heteroatoms (C2-5 heteroalkenyl). In certain embodiments, the heteroalkenyl group contains 2-4 carbon atoms and 1-2 heteroatoms (C2-4 heteroalkenyl). In certain embodiments, the heteroalkenyl group contains 2-3 carbon atoms and 1 heteroatom (C2-3 heteroalkenyl). The term “heteroalkenylene,” as used herein, refers to a biradical derived from an heteroalkenyl group, as defined herein, by removal of two hydrogen atoms. Heteroalkenylene groups may be cyclic or acyclic, branched or unbranched, substituted or unsubstituted.

The term “heteroalkynyl,” as used herein, refers to an alkynyl moiety, as defined herein, which further contains one or more heteroatoms (e.g., oxygen, sulfur, nitrogen, phosphorus, or silicon atoms) in between carbon atoms. In certain embodiments, the heteroalkynyl group contains 2-20 carbon atoms and 1-6 heteroatoms (C2-20 heteroalkynyl). In certain embodiments, the heteroalkynyl group contains 2-10 carbon atoms and 1-4 heteroatoms (C2-10 heteroalkynyl). In certain embodiments, the heteroalkynyl group contains 2-6 carbon atoms and 1-3 heteroatoms (C2-6 heteroalkynyl). In certain embodiments, the heteroalkynyl group contains 2-5 carbon atoms and 1-3 heteroatoms (C2-5 heteroalkynyl). In certain embodiments, the heteroalkynyl group contains 2-4 carbon atoms and 1-2 heteroatoms (C2-4 heteroalkynyl). In certain embodiments, the heteroalkynyl group contains 2-3 carbon atoms and 1 heteroatom (C2-3 heteroalkynyl). The term “heteroalkynylene,” as used herein, refers to a biradical derived from an heteroalkynyl group, as defined herein, by removal of two hydrogen atoms. Heteroalkynylene groups may be cyclic or acyclic, branched or unbranched, substituted or unsubstituted.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5—, 6—, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety.

The term “aryl,” as used herein, refers to an aromatic mono- or polycyclic ring system having 3-20 ring atoms, of which all the ring atoms are carbon, and which may be substituted or unsubstituted. In certain embodiments of the present disclosure, “aryl” refers to a mono, bi, or tricyclic C4-C20 aromatic ring system having one, two, or three aromatic rings which include, but are not limited to, phenyl, biphenyl, naphthyl, and the like, which may bear one or more substituents. Aryl substituents include, but are not limited to, any of the substituents described herein, that result in the formation of a stable moiety. The term “arylene,” as used herein refers to an aryl biradical derived from an aryl group, as defined herein, by removal of two hydrogen atoms. Arylene groups may be substituted or unsubstituted. Arylene group substituents include, but are not limited to, any of the substituents described herein, that result in the formation of a stable moiety. Additionally, arylene groups may be incorporated as a linker group into an alkylene, alkenylene, alkynylene, heteroalkylene, heteroalkenylene, or heteroalkynylene group, as defined herein.

The term “heteroaryl,” as used herein, refers to an aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Examples of heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety. The term “heteroarylene,” as used herein, refers to a biradical derived from an heteroaryl group, as defined herein, by removal of two hydrogen atoms. Heteroarylene groups may be substituted or unsubstituted. Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety.

The term “acyl,” as used herein, is a subset of a substituted alkyl group, and refers to a group having the general formula -C(═O)RA, -C(═O)ORA, -C(═O)—O—C(═O)RA, -C(═O)SRA, -C(═O)N(RA)₂, -C(═S)RA, -C(═S)N(RA)₂, and -C(═S)S(RA), -C(═NRA)RA, -C(═NRA)ORA, C(═NRA)SRA, and -C(═NRA)N(RA)₂, wherein RA is hydrogen; halogen; substituted or unsubstituted hydroxyl; substituted or unsubstituted thiol; substituted or unsubstituted amino; acyl; optionally substituted aliphatic; optionally substituted heteroaliphatic; optionally substituted alkyl; optionally substituted alkenyl; optionally substituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di heteroarylamino; or two RA groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety.

The term “acylene,” as used herein, is a subset of a substituted alkylene, substituted alkenylene, substituted alkynylene, substituted heteroalkylene, substituted heteroalkenylene, or substituted heteroalkynylene group, and refers to an acyl group having the general formulae: -R₀-(C═X₁)-R₀-, -R-X₂(C═X₁)-R₀-, or-R₀-X₂(C═X₁)X₃-R₀-, where X₁, X₂, and X₃ is, independently, oxygen, sulfur, or NRr, wherein Rr is hydrogen or optionally substituted aliphatic, and Rois an optionally substituted alkylene, alkenylene, alkynylene, heteroalkylene, heteroalkenylene, or heteroalkynylene group, as defined herein. Exemplary acylene groups wherein Rois alkylene includes-(CH₂)T-O(C═O)-(CH₂)T-; -(CH₂)T-NRr(C═O)-(CH₂)T-; -(CH₂)T-O(C═NRr)-(CH₂)T-; -(CH₂)T-NRr(C═NRr)-(CH₂)T-; -(CH₂)T-(C═O)-(CH₂)T-; -(CH₂)T-(C-NRr)-(CH₂)T-; -(CH₂)T-S(C═S)-(CH₂)T-; -(CH₂)T-NRr(C═S)-(CH₂)-; -(CH₂)T-S(C═NRr)-(CH₂)T-; -(CH₂)T-O(C═S)-(CH₂)T-; -(CH₂)T-(C═S)-(CH₂)T-; or-(CH₂)T-S(C═O)-(CH₂)T-, and the like, which may bear one or more substituents; and wherein each instance of T is, independently, an integer between 0 to 20. Acylene substituents include, but are not limited to, any of the substituents described herein, that result in the formation of a stable moiety.

The term “amino,” as used herein, refers to a group of the formula (—NH₂). A “substituted amino” refers either to a mono-substituted amine (—NHRh) of a disubstituted amine (—NRh₂), wherein the Rh substituent is any substituent as described herein that results in the formation of a stable moiety (e.g., an amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the Rh substituents of the di-substituted amino group (—NRh₂) form a 5- to 6-membered heterocyclic ring.

The term “hydroxy” or “hydroxyl,” as used herein, refers to a group of the formula (—OH). A “substituted hydroxyl” refers to a group of the formula (—ORi), wherein Ri can be any substituent which results in a stable moiety (e.g., a hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “thio” or “thiol,” as used herein, refers to a group of the formula (—SH). A “substituted thiol” refers to a group of the formula (—SRr), wherein Rr can be any substituent that results in the formation of a stable moiety (e.g., a thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “imino,” as used herein, refers to a group of the formula (═NRr), wherein Rr corresponds to hydrogen or any substituent as described herein, that results in the formation of a stable moiety (for example, an amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted).

The term “azide” or “azido,” as used herein, refers to a group of the formula (—N₃).

### III. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be a cell-free sample (e.g., serum, plasma). The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be a sample comprising cell-free nucleic acid. Cell-free nucleic acid includes, for example, cell-free DNA (cfDNA) and cell-free RNA (cfRNA). Cell-free nucleic acid may be isolated, extracted, or otherwise purified from a biological sample for further analysis or processing using the methods and compositions disclosed herein. In some aspects, a sample comprises at least, at most, or about 1000, 900, 800, 700, 600, 500, 400, 300, 200, 100, 50, 40, 30, 20, 10, 5, 4, or 3 ng of nucleic acid, or any range or value derivable therein. In some aspects, a sample comprises at most 50 ng of DNA (e.g., cfDNA). In some aspects, a sample comprises at most 50 ng of RNA (e.g., cfRNA). As disclosed herein, certain methods of the present disclosure, including methods for modifying, analyzing, and sequencing 5mC, are particularly suitable for processing and analysis of samples having low amounts of nucleic acid (e.g., less than 200, 150, 100, 50, 30, 20, or 10 ng of DNA and/or RNA).

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple tissue samples may be obtained for diagnosis by the methods described herein. In other cases, multiple samples, such as one or more samples from one tissue type and one or more samples from another specimen may be obtained for diagnosis by the methods. In some cases, multiple samples such as one or more samples from one tissue type and one or more samples from another specimen may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Additional Assay Methods

### A. Detection of Methylated DNA

Aspects of the methods include assaying nucleic acids to determine expression levels and/or methylation levels of nucleic acids. The present disclosure provides certain methods and compositions for bisulfute-free, single base-resolution sequencing of methylated DNA, as described in more detail elsewhere herein. Certain additional assays for the detection and analysis of methylated DNA are known in the art, examples of which are described below.

### 1. HPLC-UV

The technique of HPLC-UV (high performance liquid chromatography-ultraviolet), developed by Kuo and colleagues in 1980 (described further in Kuo K.C. et al., Nucleic Acids Res. 1980; 8:4763-4776, which is herein incorporated by reference) can be used to quantify the amount of deoxycytidine (dC) and methylated cytosines (5mC) present in a hydrolysed DNA sample. The method includes hydrolyzing the DNA into its constituent nucleoside bases, the 5 mC and dC bases are separated chromatographically and, then, the fractions are measured.

Then, the 5 mC/dC ratio can be calculated for each sample, and this can be compared between the experimental and control samples.

### 2. LC-MS/MS

Liquid chromatography coupled with tandem mass spectrometry (LC-MS/MS) is an high-sensitivity approach to HPLC-UV, which requires much smaller quantities of the hydrolysed DNA sample. In the case of mammalian DNA, of which ~2%-5% of all cytosine residues are methylated, LC-MS/MS has been validated for detecting levels of methylation levels ranging from 0.05%-10%, and it can confidently detect differences between samples as small as ~0.25% of the total cytosine residues, which corresponds to ~5% differences in global DNA methylation. The procedure routinely requires 50-100 ng of DNA sample, although much smaller amounts (as low as 5 ng) have been successfully profiled. Another major benefit of this method is that it is not adversely affected by poor-quality DNA (e.g., DNA derived from FFPE samples).

### 3. ELISA-Based Methods

There are several commercially available kits, all enzyme-linked immunosorbent assay (ELISA) based, that enable the quick assessment of DNA methylation status. These assays include Global DNA Methylation ELISA, available from Cell Biolabs; Imprint Methylated DNA Quantification kit (sandwich ELISA), available from Sigma-Aldrich; EpiSeeker methylated DNA Quantification Kit, available from abcam; Global DNA Methylation Assay-LINE-1, available from Active Motif; 5-mC DNA ELISA Kit, available from Zymo Research; MethylFlash Methylated DNA5-mC Quantification Kit and MethylFlash Methylated DNA5-mC Quantification Kit, available from Epigentek.

Briefly, the DNA sample is captured on an ELISA plate, and the methylated cytosines are detected through sequential incubations steps with: (1) a primary antibody raised against 5 Mc; (2) a labelled secondary antibody; and then (3) colorimetric/fluorometric detection reagents.

The Global DNA Methylation Assay-LINE-1 specifically determines the methylation levels of LINE-1 (long interspersed nuclear elements-1) retrotransposons, of which ~17% of the human genome is composed. These are well established as a surrogate for global DNA methylation. Briefly, fragmented DNA is hybridized to biotinylated LINE-1 probes, which are then subsequently immobilized to a streptavidin-coated plate. Following washing and blocking steps, methylated cytosines are quantified using an anti-5 mC antibody, HRP-conjugated secondary antibody and chemiluminescent detection reagents. Samples are quantified against a standard curve generated from standards with known LINE-1 methylation levels. The manufacturers claim the assay can detect DNA methylation levels as low as 0.5%. Thus, by analysing a fraction of the genome, it is possible to achieve better accuracy in quantification.

### 4. LINE-1 Pyrosequencing

Levels of LINE-1 methylation can alternatively be assessed by another method that involves the bisulfite conversion of DNA, followed by the PCR amplification of LINE-1 conservative sequences. The methylation status of the amplified fragments is then quantified by pyrosequencing, which is able to resolve differences between DNA samples as small as ~5%. Even though the technique assesses LINE-1 elements and therefore relatively few CpG sites, this has been shown to reflect global DNA methylation changes very well. The method is particularly well suited for high throughput analysis of cancer samples, where hypomethylation is very often associated with poor prognosis. This method is particularly suitable for human DNA, but there are also versions adapted to rat and mouse genomes.

### 5. AFLP and RFLP

Detection of fragments that are differentially methylated could be achieved by traditional PCR-based amplification fragment length polymorphism (AFLP), restriction fragment length polymorphism (RFLP) or protocols that employ a combination of both.

### 6. LUMA

The LUMA (luminometric methylation assay) technique utilizes a combination of two DNA restriction digest reactions performed in parallel and subsequent pyrosequencing reactions to fill-in the protruding ends of the digested DNA strands. One digestion reaction is performed with the CpG methylation-sensitive enzyme HpaII; while the parallel reaction uses the methylation-insensitive enzyme MspI, which will cut at all CCGG sites. The enzyme EcoRI is included in both reactions as an internal control. Both MspI and HpaII generate 5′-CG overhangs after DNA cleavage, whereas EcoRI produces 5′-AATT overhangs, which are then filled in with the subsequent pyrosequencing-based extension assay. Essentially, the measured light signal calculated as the HpaII/MspI ratio is proportional to the amount of unmethylated DNA present in the sample. As the sequence of nucleotides that are added in pyrosequencing reaction is known, the specificity of the method is very high and the variability is low, which is essential for the detection of small changes in global methylation. LUMA requires only a relatively small amount of DNA (250-500 ng), demonstrates little variability and has the benefit of an internal control to account for variability in the amount of DNA input.

### 7. Bisulfite Sequencing

The bisulfite treatment of DNA mediates the deamination of cytosine into uracil, and these converted residues will be read as thymine, as determined by PCR-amplification and subsequent Sanger sequencing analysis. However, 5mC residues are resistant to this conversion and, so, will remain read as cytosine. Thus, comparing the Sanger sequencing read from an untreated DNA sample to the same sample following bisulfite treatment enables the detection of the methylated cytosines. With the advent of next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5 mC-enriched fraction of the genome is not only a less expensive approach, but it also allows one to increase the sequencing coverage and, therefore, precision in revealing differentially-methylated regions. Sequencing could be done using any existing NGS platform; Illumina and Life Technologies both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after MspI digestion that recognizes CCGG sites (and it cut both methylated and unmethylated sites). It ensures isolation of ~85% of CpG islands in the human genome. Then, the same bisulfite conversion and library preparation is performed as for WGBS. The RRBS procedure normally requires ~100 ng-1 μg of DNA.

As disclosed herein, certain methods of the disclosure do not comprise bisulfite sequencing. In certain aspects, a method of the disclosure does not include any treatment, incubation, or mixture with bisulfite (e.g., sodium bisulfite, ammonium bisulfite, or other bisulfite source).

### 8. Array or Bead Hybridization

Methylated DNA fractions of the genome, usually obtained by immunoprecipitation, could be used for hybridization with microarrays. Currently available examples of such arrays include: the Human CpG Island Microarray Kit (Agilent), the GeneChip Human Promoter 1.OR Array and the GeneChip Human Tiling 2.0R Array Set (Affymetrix).

The search for differentially-methylated regions using bisulfite-converted DNA could be done with the use of different techniques. Some of them are easier to perform and analyse than others, because only a fraction of the genome is used. The most pronounced functional effect of DNA methylation occurs within gene promoter regions, enhancer regulatory elements and 3′ untranslated regions (3′UTRs). Assays that focus on these specific regions, such as the Infinium HumanMethylation450 Bead Chip array by Illumina, can be used. The arrays can be used to detect methylation status of genes, including miRNA promoters, 5′ UTR, 3′ UTR, coding regions (~17 CpG per gene) and island shores (regions ~2 kb upstream of the CpG islands).

Briefly, bisulfite-treated genomic DNA is mixed with assay oligos, one of which is complimentary to uracil (converted from original unmethylated cytosine), and another is complimentary to the cytosine of the methylated (and therefore protected from conversion) site. Following hybridization, primers are extended and ligated to locus-specific oligos to create a template for universal PCR. Finally, labelled PCR primers are used to create detectable products that are immobilized to bar-coded beads, and the signal is measured. The ratio between two types of beads for each locus (individual CpG) is an indicator of its methylation level.

It is possible to purchase kits that utilize the extension of methylation-specific primers for validation studies. In the VeraCode Methylation assay from Illumina, 96 or 384 user-specified CpG loci are analysed with the GoldenGate Assay for Methylation. Differently from the BeadChip assay, the VeraCode assay requires the BeadXpress Reader for scanning.

### 9. Methyl-Sensitive Cut Counting: Endonuclease Digestion Followed by Sequencing

As an alternative to sequencing a substantial amount of methylated (or unmethylated) DNA, one could generate snippets from these regions and map them back to the genome after sequencing. Moreover, coverage in NGS could be good enough to quantify the methylation level for particular loci. The technique of serial analysis of gene expression (SAGE) has been adapted for this purpose and is known as methylation-specific digital karyotyping, as well as a similar technique, called methyl-sensitive cut counting (MSCC).

In summary, in all of these methods, methylation-sensitive endonuclease(s), e.g., HpaII is used for initial digestion of genomic DNA in unmethylated sites followed by adaptor ligation that contains the site for another digestion enzyme that is cut outside of its recognized site, e.g., EcoP15I or MmeI. These ways, small fragments are generated that are located in close proximity to the original HpaII site. Then, NGS and mapping to the genome are performed. The number of reads for each HpaII site correlates with its methylation level.

Recently, a number of restriction enzymes have been discovered that use methylated DNA as a substrate (methylation-dependent endonucleases). Most of them were discovered and are sold by SibEnzyme: BisI, BlsI, GlaI. GluI, Krol, Mtel, PcsI, PkrI. The unique ability of these enzymes to cut only methylated sites has been utilized in the method that achieved selective amplification of methylated DNA. Three methylation-dependent endonucleases that are available from New England Biolabs (FspEI, MspJI and LpnPI) are type IIS enzymes that cut outside of the recognition site and, therefore, are able to generate snippets of 32 bp around the fully-methylated recognition site that contains CpG. These short fragments could be sequences and aligned to the reference genome. The number of reads obtained for each specific 32-bp fragment could be an indicator of its methylation level. Similarly, short fragments could be generated from methylated CpG islands with Escherichia coli's methyl-specific endonuclease McrBC, which cuts DNA between two half-sites of (G/A) mC that are lying within 50 bp-3000 bp from each other.

### B. Sequencing

DNA, including DNA comprising a nucleobase derivative (e.g., N₃-T) could be used for the amplification of the region of interest followed by sequencing. Primers are designed around the CpG island and used for PCR amplification of DNA. The resulting PCR products could be cloned and sequenced. Accordingly, aspects of the disclosure may include sequencing nucleic acids to detect methylation of nucleic acids and/or biomarkers. In some embodiments, the methods of the disclosure include a sequencing method. Exemplary sequencing methods include those described below.

### 1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

### 2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLID platform, which is now owned by Life Technologies.

### 3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLID on the other.

### 4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera. Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1x coverage per hour per instrument, and one human genome re-sequenced (at approx. 30x) per day per instrument (equipped with a single camera).

### 5. Solid Sequencing.

Applied Biosystems' (now a Thermo Fisher Scientific brand)SOLID technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

### 6. Ion Torrent Semiconductor Sequencing.

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### 7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

### 8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### 9. Single Molecule Real Time (SMRT) Sequencing.

SMRT sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.]

### C. Additional Assay Methods

In some embodiments, methods involve amplifying and/or sequencing one or more target genomic regions using at least one pair of primers specific to the target genomic regions. In certain embodiments, the primers are heptamers. In other embodiments, enzymes are added such as primases or primase/polymerase combination enzyme to the amplification step to synthesize primers.

In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be or may be at least 50, 100, 200, 300, 400, 500 or more probes/cm² (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

### V. Clinical and Diagnostic Applications

The methods of the disclosure may be useful for evaluating nucleic acid (e.g., DNA, RNA) for clinical, diagnostic, or research purposes. Certain embodiments relate to a method for evaluating a sample comprising DNA molecules. Further aspects relate to a method for evaluating a sample comprising RNA molecules. The evaluation may be the detection or determination of a particular nucleotide, such as 5-methylcytosine (5mC).

A sample may include but is not limited to, tissue, cells, or biological material from cells or derived from cells of a subject. In some embodiments, the sample comprises cell-free DNA. In some embodiments, the sample comprises a fertilized egg, a zygote, a blastocyst, or a blastomere. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

In some embodiments, the methods of the disclosure can be used in the discovery of novel biomarkers for a disease or condition. In some embodiments, the methods of the disclosure can performed on a sample from a patient to provide a prognosis for a certain disease or condition in the patient. In some embodiments, the methods of the disclosure can be performed on a sample from a patient to predict the patient's response to a particular therapy. In some embodiments, the disease comprises a cancer. In some embodiments, the cancer comprises ovarian, prostate, colon, or lung cancer. In some embodiments, the method is for determining novel biomarkers for ovarian, prostate, colon, or lung cancer by evaluating cell-free nucleic acid (e.g., cell-free DNA) using methods of the disclosure. In some embodiments, the methods of the disclosure may be used on fetal DNA isolated from a pregnant female. In some embodiments, the methods of the disclosure may be used for prenatal diagnostics using fetal DNA isolated from a pregnant female.

### VI. Detecting a Genetic Signature

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. Single Nucleotide Polymorphism (SNP) Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a

C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34,3 5, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

### B. Copy Number Variation Detection

Particular embodiments of the disclosure concern methods of detecting a copy number variation (CNV) of a particular allele. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array, wherein the array is capable of detecting CNVs on the entire X chromosome and/or all targets of miR-362. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some embodiments, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some embodiments, the CNV can be detected using nucleic acid sequencing. Sequencing techniques that could be used include, but are not limited to, whole genome sequencing, whole exome sequencing, and/or targeted sequencing.

### C. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20x, 25x, 30x, 35x, 40x, 45x, 50x, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### D. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

### E. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

### VII. Kits

Certain aspects of the present disclosure also concern kits containing compositions of the disclosure or compositions to implement methods disclosed herein. In some embodiments, disclosed are kits that can be used to modify and/or detect 5mC in a target DNA. Each kit may also include additional components that are useful for purifying, amplifying, or sequencing the DNA, or for other applications of the present disclosure as described herein.

In some embodiments, a kit of the disclosure comprises instructions for use. In some embodiments, the instructions include instructions for incubating a nucleic acid molecule (e.g., an RNA molecule or a DNA molecule) with an ageng (e.g., a TET enzyme) under conditions sufficient to oxidize 5mC in a DNA sample to 5-carboxylcytosine (5caC) or 5-formylcytosine (5fC). In some embodiments, the instructions include instructions for incubating a nucleic acid molecule with a TDG enzyme to excise the 5caC or 5fC creating an abasic site. In some embodiments, the instructions include instructions for incubating a nucleic acid molecule comprising an abasic site with a nucleobase derivative (e.g., a thymine derivative such as N₃-T) to attach the nucleobase derivative to the nucleic acid molecule at the abasic site. Such instructions may include instructions for providing the conditions necessary for modification of all 5mC on the nucleic acid molecule(s). Such conditions may include, for example, pH conditions, temperature conditions, incubation time, etc. Examples of such conditions are disclosed herein. In some embodiments, the instructions comprise instructions for incubating the nucleic acid molecule for, for at most, or for at least 12, 11, 10, 9, 8, 7, 6, 5, 4, 3, 2, or 1 hours, or any range or value derivable therein, with a TET enzyme, TDG enzyme, and/or nucleobase derivative. In some embodiments, the instructions comprise instructions for incubating the nucleic acid molecule at a temperature of between about 25° C. and about 45° C. In some embodiments, the instructions comprise instructions for incubating the nucleic acid molecule at a temperature of about 25, 26, 27, 28, 29, 30, 31, 32, 33, 34, 35, 36, 37, 38, 39, 40, 41, 42, 43, 44, or 45° C., or any range or value derivable therein. In some embodiments, the instructions comprise instructions for incubating the nucleic acid molecule at a temperature of about 37° C. In some embodiments, the instructions comprise instructions for incubating the nucleic acid molecule at a temperature of 37° C.

In some embodiments, the kit comprises a reverse transcriptase (RT) enzyme. In some embodiments, the RT enzyme is AMV RT, MMLV RT, SuperScript III, or SuperScript IV. In some embodiments, the reverse transcriptase enzyme is SuperScript IV.

In some embodiments, the kit comprises a TET enzyme. In some embodiments, the TDG enzyme is a mammalian or murine TET enzyme. In some aspects, the TET enzyme is a TET1, TET2, or TET3 enzyme. In some embodiments, the kit comprises a TDG enzyme. In some embodiments, the TDG enzyme is a mammalian or murine TDG enzyme.

In some embodiments, the kit comprises a nucleobase derivative. In some embodiments, the nucleobase derivative is an adenine derivative. In some embodiments, the nucleobase derivative is a thymine derivative. In some embodiments, the thymine derivative is a compound of formula (I):

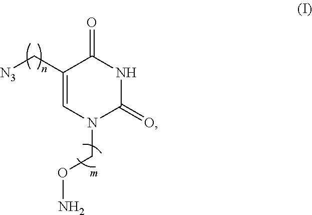

wherein n is an integer from 1 to 5 and m is an integer from 1 to 5. In some aspects, n is 1, 2, 3, 4, or 5. In some aspects, m is 1, 2, 3, 4, or 5. In some aspects, the thymine derivative is N₃-T:

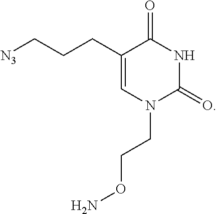 [SMILES: [N-]=[N+]=NCCCc1cn(CCON)c(=O)[nH]c1=O]

The kit may optionally provide additional components that are useful in the procedure. These optional components include buffers, capture reagents, developing reagents, labels, reacting surfaces, means for detection, control samples, instructions, and interpretive information. In certain embodiments, a kit contains, contains at least, or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

In some embodiments, the kit does not comprise a bisulfite source (e.g., sodium bisulfite, ammonium bisulfite, etc.).

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1x, 2x, 5x, 10x, or 20× or more.

In certain aspects, negative and/or positive control nucleic acids, probes, and inhibitors are included in some kit embodiments. In addition, a kit may include a sample that is a negative or positive control, for example a nucleic acid that does not comprise a 5mC may be included as a negative control and a nucleic acid that does comprise a 5mC may be included as a positive control.

It is specifically contemplated that a kit of the present disclosure may exclude any one or more of the described components in certain embodiments.

### Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1-Development and Analysis of a New Bisulfite-Free Direct Sequencing of 5-Methylcytosine at Base Resolution

Currently, bisulfite sequencing is considered the “gold standard” for DNA methylation analysis. The bisulfite sequencing method, including its derivative methods such as oxidative bisulfite sequencing (oxBS), are all based on bisulfite treatment to convert unmethylated cytosine to uracil while leaving 5mC intact. However, the harsh treatment causes severe DNA degradation since this reaction is run under acidic conditions with heating to 64° C. for 2.5 hrs. Therefore, these methods may not be ideally suited for low input DNA such as cfDNA samples. In the meantime, because the bisulfite method turns all unmodified cytosine to uracil, which constitutes more than 95% of total genomic cytosine, the complexity of the sequence will be affected severely, leading to a high sequencing depth requirement.

Disclosed herein is as a novel strategy that can address all these challenges and achieve base-resolution 5mC sequencing without involving toxic chemicals, described in some aspects herein as “TET/TDG-mediated 5mC labeling and sequencing” (also “TT-5mC-seq”) (FIG. 1A). Included in the disclosed methods is an option to add an enrichment step of 5mC-containing fragments to significantly reduce costs if needed. The active demethylation process of 5mC is leveraged, using TET to oxidize 5mC to 5caC.

In most cases the level of 5hmC on genomic DNA is so low that its potential presence does not interfere with 5mC measurement. However, if 5hmC level is high at particular sites, one can perform selective sequencing of just 5mC by applyingβ-glucosyltransferase to first label 5hmC with glucose, which protects 5hmC from the TET-mediated oxidation and following steps (FIG. 1B).

Then, TDG is used to excise 5caC and create an abasic site, which can be further modified by a thymine mimic almost stoichiometrically. Therefore, only 5mC will be read as T with unmodified C read as C in subsequent high-throughput sequencing. This procedure is mild and avoids all chemicals. In addition, with the tethered azide group (N₃-T) attached to thymine mimic, the method can use bio-orthogonal chemistry to add a biotin or other affinity tag for optional enrichment and sequencing.

### General Design of TT-5mC-Seq

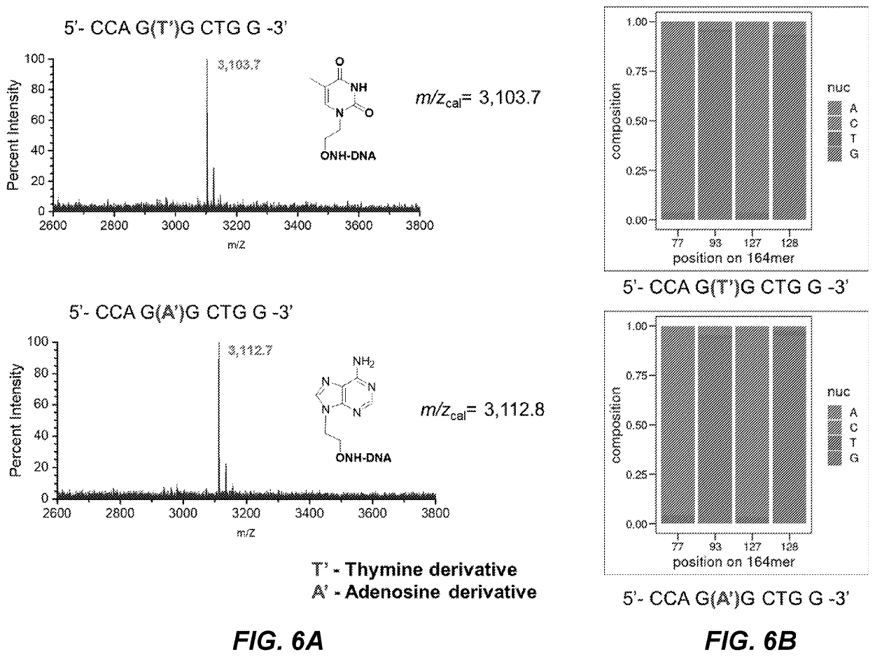

Initially, in one aspect, murine TET1 (mTET1) enzyme was used to oxide 5mC to 5caC with high efficiency. The inventors then took advantage of the base excision repair (BER) process of TDG to generate an abasic site at the 5caC site (derived from 5mC oxidation). Next, a chemically synthesized thymine mimic (N₃-T) was prepared from commercial reagents and introduced following six steps (FIG. 5). This compound consists of three parts (FIG. 1B). The hydroxylamine functional group was designed to react with the aldehyde group in the abasic site for selective labeling. Subsequently, the thymine nucleobase attached to the abasic site will be read as T in the following PCR amplification step, therefore leading to a C-to-T mutation to achieve the base-resolution sequencing. The azide tether added serves as an optional bioorthogonal handle. One can either directly sequence the labeled DNA and thus detect not only 5mC but also its stoichiometry based on mutation frequency, or one can employ a dibenzocyclooctyne-modified biotin (DBCO-biotin) and induce biotinylation of the previously methylated DNA fragment via click chemistry. The enriched DNA can be amplified for sequencing. Note that as a thymine derivative, N₃-T can be recognized by DNA polymerase as T during PCR process. Therefore, TT-5mC-seq induced 5mC-to-T mutation to identify 5mC sites at single-base resolution. The method can include addition of thymine mimic with or without N₃ (with or without enrichment). Other base mimics were also synthesized with hydroxylamine or related groups to induce C-to-A mutation or C-to-T mutations without azide modification, in addition to C-to-T mutations with azide (FIGS. 6A and 6B).

### Optimization of the Reaction Conditions Using DNA Model Oligos by MALDI-TOF MS

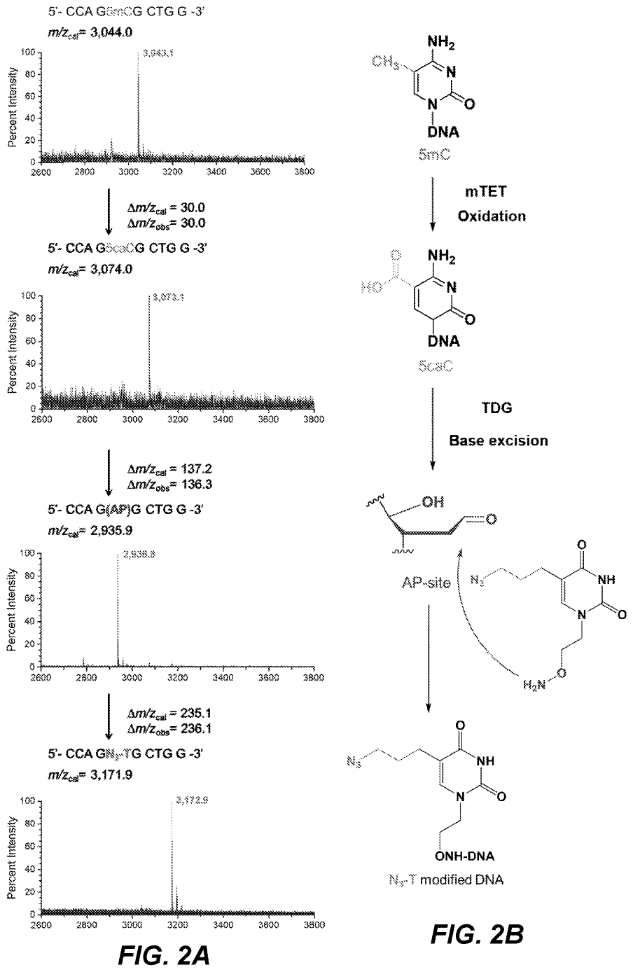

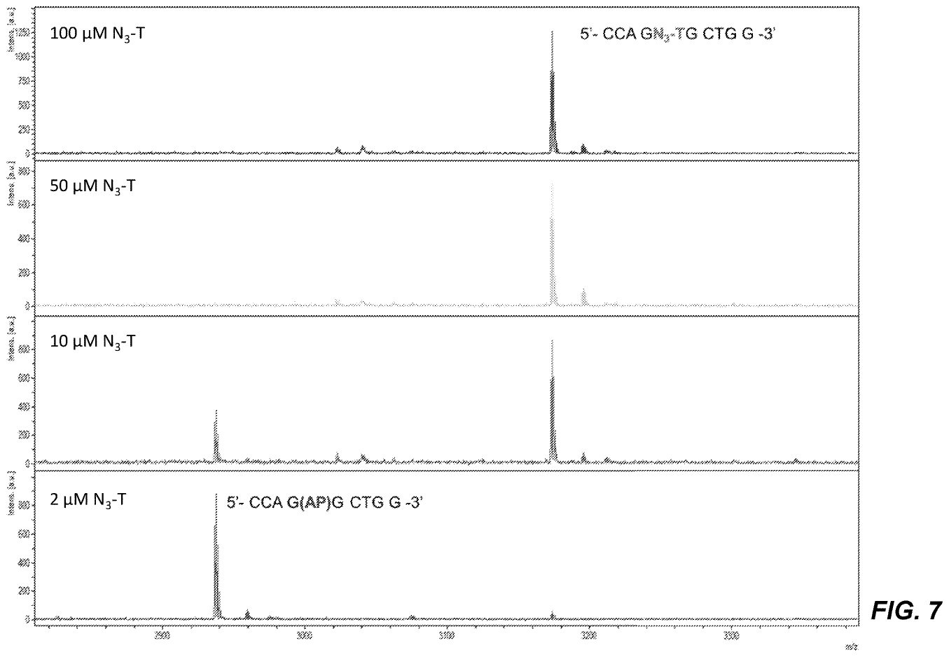

For TDG excision, turnover rate of the excision reaction has long been a big problem. To achieve complete transformation from 5caC to the abasic site, a 10-mer model 5caC modified DNA was mixed with TDG in different concentrations. It was found that found that a 10-fold molar ratio of TDG (100 nM TDG for 10 nM DNA substrate) could completely excise 5caC to afford the AP site. The reactions were performed at 22° C. for 60 min in reaction buffer containing 25 mM HEPES, pH 7.4, 0.5 mM EDTA, 0.5 mg/mL BSA, and 0.5 mM DTT. Next the reaction between the abasic site and the thymine mimic (N₃-thymine) by mixing 100 ng 10-mer model DNA in water with N₃-T in DMSO under different conditions (FIG. 7). After reacting with 50 mM N₃-T in MES buffer (pH=6.0) at 37° C. for 2 h, a complete conversion of the AP site to the expected product was observed based on the matrix-assisted laser desorption/ionization-time of flight (MALDI-TOF) mass spectrum analysis. With this method, the entire TT-5mC-seq process was validated by MALDI-TOF (FIGS. 2A and 2B).

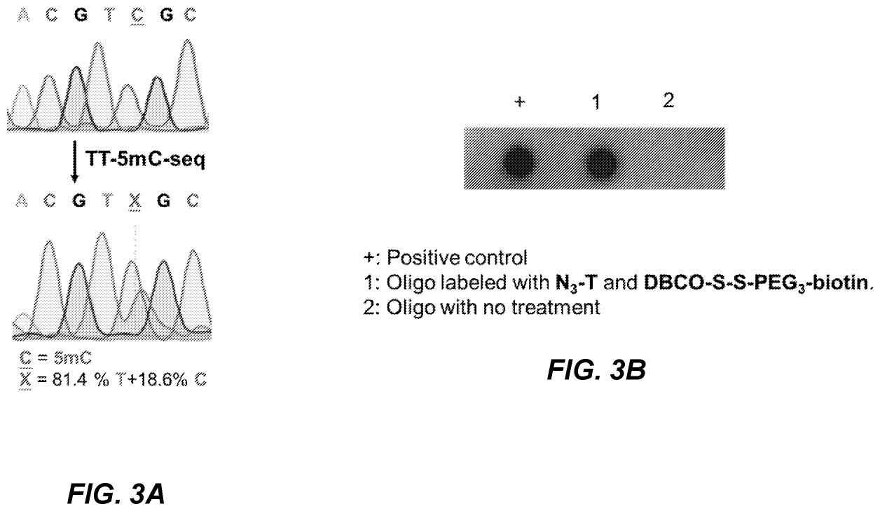

Next an 82-mer synthetic DNA oligo was prepared containing both C and 5mC. After TT-5mC-seq treatment, Sanger sequencing results showed that 5mC was read as T with a high conversion rate (81.4%) (FIG. 3A), whereas, C remained unchanged after treatment, confirming that our optimized condition is highly efficient to induce 5mC-to-T conversion. The undesired C-to-U conversion can be completely avoided. A dot blot assay was also run to further verify the method can apply to further enrichment by click reaction, and according to the result, the whole process was robust and repeatable (FIG. 3B).

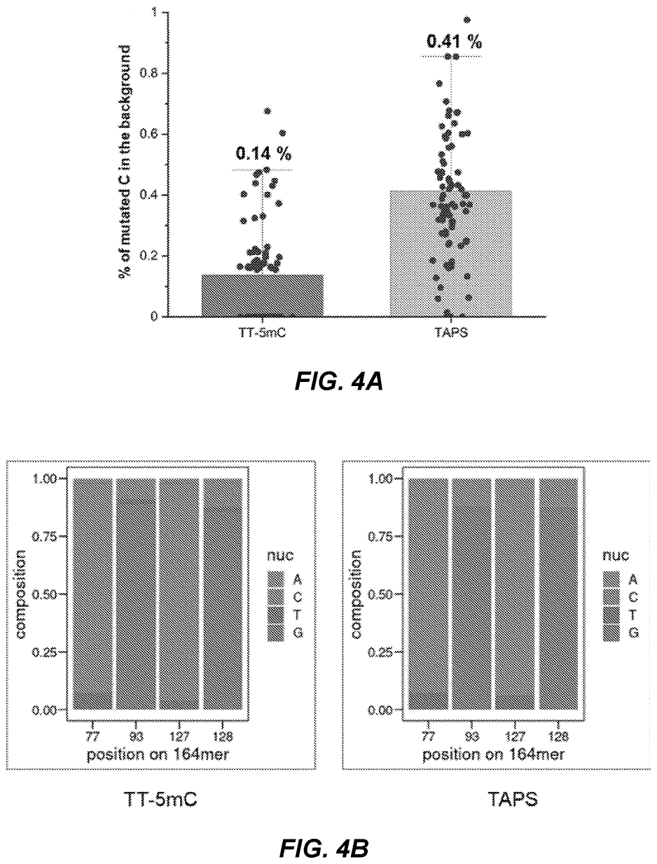

With the above-established principle, the inventors proceeded to compare the new method to TET-assisted pyridine borane sequencing (TAPS-seq), a method described in Liu, Yibin, et al. “Bisulfite-free direct detection of 5-methylcytosine and 5-hydroxymethylcytosine at base resolution.” Nature biotechnology 37.4 (2019): 424-429, incorporated herein by reference in its entirety. The comparison was performed using a longer 164-mer synthetic spike-in (mimicing the size of cfDNA) that contained two 5mC modification sites on each strand. After different treatments and library construction using KAPA Hyper Plus Kits for NGS DNA Library Prep Kit (double-stranded DNA library construction) and NGS sequencing, sequencing data showed that the disclosed method gave a mutation rate comparable to TAPS-seq on all four mutation sites while reducing the background noise (undesired C-to-DHU conversion) by 66% (FIG. 4).

In summary, described is the development and demonstration of a novel strategy, termed TT-5mC-seq, for whole-genome localization of 5mC at single-base resolution. By utilizing TET/TDG treatment and N₃-T labeling, the method creates 5mC-to-T conversion under mild conditions, which outperforms traditional bisulfite-treat-based methods in providing direct readout of 5mC with much less damage to DNA samples. The new method also overcomes the undesired background problem in TAPS-seq. In addition, this approach can introduce an azide modification and achieve enrichment of 5mC-containing DNA fragments.

### Example 2—Synthesis of N₃-T

Synthesis of compound 3: To a solution of 5-iodouracil (1, 1.0 g, 4.2020 mmol) in a mixture of pyridine and acetonitrile (2/5, v/v, 21 mL) was added Benzoyl chloride (2, 1.5 mL, 12.606 mmol). The reaction mixture was stirred for 24 h at room temperature. After evaporation of all the volatiles, the residue was purified by silica gel column chromatography (eluting with 1:1 hexanes/ethyl acetate) to give compound 3 (1.440 g, quant.) as a white foam. ¹H NMR (400 MHz, DMSO) δ 11.93 (s, 1H), 8.15 (s, 1H), 7.99 (dd, J=8.4, 1.3 Hz, 2H), 7.84-7.74 (m, 1H), 7.60 (dd, J=8.3, 7.4 Hz, 2H). ¹³C NMR (101 MHz, DMSO) δ 169.75, 160.61, 150.31, 148.36, 136.03, 131.40, 130.91, 129.98, 67.15. HRMS C₁₁H₇IN₂NaO₃⁺ [M+Na]⁺calculated 364.9394, found 364.9389.

Synthesis of compound 5: To a stirred mixture solution of compound 3 (1.368 g, 4.0 mmol) and N-(2-bromoethoxy) phthalimide (4, 1.080 g, 4.0 mmol) in DMSO(15 mL) was added potassium carbonate (552 mg, 4 mmol). The resulting mixture was stirred for 3 h at room temperature before being diluted with water. The mixture was extracted by ethyl acetate and the combined organic layers were washed with brine three times, dried over anhydrous sodium sulfate. Filtered and concentrated. The crude product was purified by flash column chromatography (eluting with 1:1 hexanes/acetone) to afford compound 5 (1.531 g, 90%) as a white foam. ¹H NMR (400 MHz, DMSO) δ 8.57 (s, 1H), 8.06 (d, J=7.8 Hz, 2H), 7.90 (dtt, J=8.8, 5.8, 3.7 Hz, 4H), 7.80 (t, J=7.4 Hz, 1H), 7.62 (t, J=7.7 Hz, 2H), 4.41 (t, J=5.0 Hz, 2H), 4.14 (t, J=5.1 Hz, 2H). ¹³C NMR (101 MHz, DMSO) δ 169.24, 163.69, 160.26, 151.84, 149.87, 136.14, 135.29, 131.20, 131.03, 130.02, 129.12, 123.81, 75.78, 67.22, 47.65. HRMS C₂₁H₁₅IN₃₀₆⁺ [M+H]⁺calculated 532.0000, found 532.0005.

Synthesis of compound 7: To a 25 mL sealed tube was added compound 5 (642 mg, 1.208 mmol), CuI (23 mg, 0.121 mmol), Pd (PPh₃)₄ (70 mg, 0.061 mmol) and degassed DMF (5 mL). The resulting solution was sequentially added 2-Propyn-1-ol (6, 140 μL, 2.417 mmol) and NEt₃ (353 μL, 2.537 mmol). The reaction mixture was avoided light and stirred for 24 h at room temperature. After that, the reaction mixture was quenched with water and extracted by ethyl acetate. The combined organic layers were washed with brine three times, dried over anhydrous sodium sulfate, and concentrated in vacuo. The crude product was purified by flash column chromatography (eluting with 1:1 hexanes/acetone) to afford product 7 (474 mg, 86%) as a white foam. ¹H NMR (400 MHz, CDCl₃) δ 8.06 (d, J=7.1 Hz, 2H), 8.01 (s, 1H), 7.87 (dd, J=5.5, 3.1 Hz, 2H), 7.80 (dd, J=5.5, 3.1 Hz, 2H), 7.66 (dd, J=7.5, 2.0 Hz, 2H), 7.52 (t, J=7.7 Hz, 2H), 4.46 (t, J=4.5 Hz, 2H), 4.42 (d, J=0.9 Hz, 2H), 4.15 (t, J=4.5 Hz, 2H). 13C NMR (101 MHz, CDCl₃) δ 167.83, 163.34, 161.28, 148.99, 148.48, 135.25, 134.97, 131.15, 130.84, 129.24, 128.61, 123.87, 99.24, 92.81, 76.33, 51.51, 48.62. HRMS C₂₄H₁₈N₃O₇⁺ [M+H]⁺calculated 460.1139, found 460.1138.

Synthesis of compound 8: To a stirred solution of compound 7 (280 mg, 0.61 mmol) in a mixture of MeOH and acetone (20/1, v/v, 21 mL) was added Pd/C (28 mg, 10% wt). The resulting mixture was added a H₂ balloon and stirred for 30 min at room temperature. The mixture was filtered and evaporated all the volatiles. The crude product was purified by flash column chromatography (eluting with 1:1 hexanes/acetone) to afford compound 8 (122 mg, 44%) as a white foam. ¹H NMR (400 MHz, CDCl₃) δ 8.06-7.99 (m, 1H), 7.86 (dd, J=5.5, 3.1 Hz, 1H), 7.79 (dd, J=5.5, 3.1 Hz, 1H), 7.64 (d, J=6.7 Hz, 1H), 7.51 (t, J=7.8 Hz, 1H), 4.48 (t, J=4.4 Hz, 1H), 4.16-4.06 (m, 1H), 3.65 (t, J=6.0 Hz, 1H), 2.53 (t, J=7.1 Hz, 1H), 1.82 (p, J=6.6 Hz, 1H). ¹³C NMR (101 MHz, CDCl₃) δ 168.87, 163.67, 163.44, 149.81, 142.48, 135.05, 134.96, 131.54, 130.64, 129.22, 128.64, 123.85, 113.73, 60.95, 47.82, 31.67, 22.73. HRMS C₂₄H₂₂N₃O₇⁺ [M+H]⁺calculated 464.1452, found 464.1460.

Synthesis of compound 9: To a stirred solution of compound 8 (60 mg, 0.129 mmol) in CHCl₃ (5.0 mL) was cooled down to 0° C. in ice-water bath. Then the reaction solution was sequentially added NEt₃ (54 μL, 0.388 mmol) and MsCl (30 μL, 0.388 mmol). After that, the mixture was warmed to room temperature slowly. The mixture was kept stirring at room temperature for 1 h. After evaporation of all the volatiles, the residue was crude product and used directly without purification. The crude methanesulfonyl product was dissolved in DMF (3.0 mL), directly. After that, the reaction solution was added NaN₃ (64 mg, 0.980 mmol). Then the resulting mixture was warmed to 40° C. and stirred for 6 h at this temperature. After being cooled to room temperature, the reaction mixture was quenched with water and extracted by ethyl acetate. The combined organic layers were washed with brine, dried over anhydrous sodium sulfide. Filtered and concentrated in vacuo. The crude product was purified by flash column chromatography (eluting with 1:1 hexanes/ethyl acetate) to afford compound 9 (30 mg, 61%) as a white foam. ¹H NMR (400 MHz, CDCl₃) δ 8.21 (s, 1H), 7.86 (dd, J=5.5, 3.1 Hz, 2H), 7.79 (dd, J=5.4, 3.2 Hz, 3H), 7.61 (d, J=1.0 Hz, 1H), 4.50-4.43 (m, 2H), 4.11 (t, J=4.6 Hz, 3H), 3.36 (t, J=6.7 Hz, 2H), 2.46 (t, J=7.3 Hz, 3H), 1.88 (p, J=7.0 Hz, 3H). 13C NMR (101 MHz, CDCl₃) δ 163.70, 163.48, 150.85, 142.50, 134.88, 128.67, 123.86, 112.91, 50.52, 47.41, 27.37, 24.07. HRMS C₁₇H₁₇N₆O₅⁺ [M+H]⁺calculated 385.1255, found 385.1251.

Synthesis of Azide-Thymine (N₃-T, 10): To a stirred solution of compound 9 (48 mg, 0.125 mmol) in DCM (5.0 mL) was added hydrazinium hydroxide solution (8 μL, 0.25 mmol). The reaction mixture was stirred for 1 h at room temperature. After that, the mixture was filtered with 0.2 μm filter unit. Evaporation of all the volatiles, the residue was dissolved in 0.5 mL DCM and filtered with 0.2 μm filter unit. After that, evaporation of the DCM to afford the purified product azide-thymine (N₃-T, 10, 26 mg, 82%). ¹H NMR (400 MHz, CDCl₃) δ 9.48 (s, 1H), 7.06 (s, 1H), 5.57 (s, 2H), 3.96 (dd, J=5.5, 3.9 Hz, 2H), 3.86 (dd, J=5.4, 3.9 Hz, 2H), 3.31 (t, J=6.5 Hz, 2H), 2.40 (dd, J=8.0, 6.8 Hz, 2H), 1.82 (dq, J=8.4, 6.6 Hz, 2H). 13C NMR (101 MHz, CDCl₃) δ 162.88, 150.17, 140.89, 111.90, 71.82, 49.51, 45.79, 26.40, 23.15. HRMS C₉H₁₅N₆₀₃⁺ [M+H]⁺calculated 255.1200, found 255.1201.

FIG. 5 shows an overview of the synthesis scheme for synthesis of N₃-T (“Azide-Thymine”; compound 10).

### Example 3-Methylation Analysis of Genomic DNA

The disclosed methods are applied to mESC gDNA. Libraries are built starting from 20 ng mESC gDNA using KAPA kit, then coupled with the new TT-5mC-seq strategy. 5mc in the mESC gDNA are identified and analyzed at base resolution. The methods are applied to different amounts of cfDNA (10 to 20 ng) derived from patients to further evaluate application in seeking disease biomarkers.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Klose, Robert J., and Adrian P. Bird. “Genomic DNA methylation: the mark and its mediators.” Trends in biochemical sciences 31.2 (2006): 89-97.
Li, En, and Yi Zhang. “DNA methylation in mammals.” Cold Spring Harbor perspectives in biology 6.5 (2014): a019133.
Booth, Michael J., et al. “Quantitative sequencing of 5-methylcytosine and 5-hydroxymethylcytosine at single-base resolution.” Science 336.6083 (2012): 934-937.
Vaisvila, Romualdas, et al. “EM-seq: detection of DNA methylation at single base resolution from picograms of DNA.” BioRxiv (2020): 2019-12.
Tahiliani M, Koh K P, Shen Y, et al. Conversion of 5-methylcytosine to 5-hydroxymethylcytosine in mammalian DNA by MLL partner TET1. Science (2009); 324:930-935.
Ito, Shinsuke, et al. “Tet proteins can convert 5-methylcytosine to 5-formylcytosine and 5-carboxylcytosine.” Science 333.6047 (2011): 1300-1303.
He, Yu-Fei, et al. “Tet-mediated formation of 5-carboxylcytosine and its excision by TDG in mammalian DNA.” Science 333.6047 (2011): 1303-1307.
Einav Nili, Gal-Yam, et al. “Cancer epigenetics: modifications, screening, and therapy.” Annu. Rev. Med. 59 (2008): 267-280.
Jones, Peter A., and Stephen B. Baylin. “The fundamental role of epigenetic events in cancer.” Nature reviews genetics 3.6 (2002): 415-428.
Yu, Miao, et al. “Base-resolution analysis of 5-hydroxymethylcytosine in the mammalian genome.” Cell 149.6 (2012): 1368-1380.
Liu, Yibin, et al. “Bisulfite-free direct detection of 5-methylcytosine and 5-hydroxymethylcytosine at base resolution.” Nature biotechnology 37.4 (2019): 424-429.

What is claimed is:

1. A method for modifying a 5-methylcytosine (5mC) in a nucleic acid molecule, the method comprising:
(a) incubating the nucleic acid molecule with an agent under conditions sufficient to oxidize the 5mC to 5-carboxylcytosine (5caC) or 5-formylcytosine (5fC);
(b) incubating the nucleic acid molecule with a thymine DNA glycosylase (TDG) enzyme to excise the 5caC or 5fC creating an abasic site; and
(c) incubating the nucleic acid molecule with a nucleobase derivative to attach the nucleobase derivative to the nucleic acid molecule at the abasic site.

2. The method of claim 1, wherein the nucleobase derivative comprises an azide moiety.

3. The method of claim 1, wherein the nucleobase derivative is a thymine derivative.

4. The method of claim 3, wherein the thymine derivative is a compound of formula I: 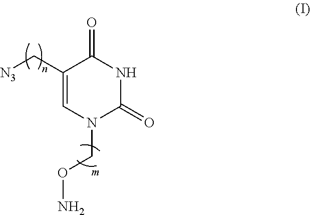
wherein n is an integer from 0 to 5 and m is an integer from 1 to 5.

5. The method of claim 4, wherein n is 3.

6. The method of claim 4, wherein m is 2.

7. The method of claim 4, wherein the thymine derivative is N₃-thymine:

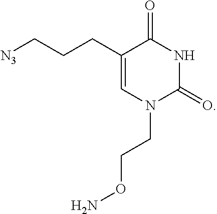 [SMILES: [N-]=[N+]=NCCCc1cn(CCON)c(=O)[nH]c1=O]

8. The method of claim 3, wherein the thymine derivative is a compound of formula (II): 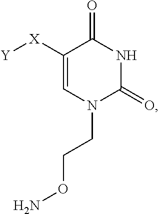
wherein X is a linker and Y is a click chemistry compatible reactive group selected from alkynes, azides, strained alkynes, dienes, dieneophiles, alkoxyamines, carbonyls, phosphines, hydrazides, thiols, alkenes, tetrazines, tetrazoles, isocyanates, isothiocyanates, and 1,3-nitrones.

9. The method of claim 8, wherein X is a C₁ to C₁₀ alkyl linker.

10. The method of claim 8, wherein X is an amide linker.

11. The method of claim 8, wherein X is an alkyl and aryl mixture linker.

12. The method of claim 8, wherein X is an alkyl and heterocycle mixture linker.

13. The method of claim 8, wherein Y is azide.

14. The method of any of claims 1-13, wherein the agent is a ten-eleven translocation (TET) enzyme.

15. The method of claim 14, wherein the TET enzyme is a mammalian TET enzyme.

16. The method of claim 14, wherein the TET enzyme is a murine TET enzyme.

17. The method of any of claims 14-16, wherein the TET enzyme is TET1.

18. The method of any of claims 14-16, wherein the TET enzyme is TET2.

19. The method of any of claims 14-16, wherein the TET enzyme is TET3.

20. The method of any of claims 1-19, wherein the TDG enzyme is a mammalian TDG enzyme.

21. The method of any of claims 1-19, wherein the TDG enzyme is a murine TDG enzyme.

22. The method of any of claims 1-21, further comprising, subsequent to (c), sequencing the nucleic acid molecule.

23. The method of any of claims 1-22, wherein the nucleic acid molecule is a deoxyribonucleic acid (DNA) molecule.

24. The method of any of claims 1-23, wherein the nucleic acid molecule was obtained from a sample comprising at or below 50 ng total nucleic acid.

25. The method of any of claims 1-24, wherein the nucleic acid molecule was obtained from a sample comprising equal to or less than 50 cells.

26. The method of any of claims 1-25, further comprising subjecting the nucleic acid molecule to a click chemistry reaction to attach a label to the nucleic acid molecule.

27. The method of claim 26, wherein the label comprises an alkyne moiety.

28. The method of claim 26 or 27, wherein the label is a dibenzocyclooctyne-modified biotin (DBCO-biotin).

29. The method of any of claims 26-28, further comprising incubating the nucleic acid molecule with streptavidin.

30. The method of any of claims 1-29, further comprising subjecting the nucleic acid molecule to a polymerase chain reaction.

31. The method of any of claims 1-30, wherein (c) is performed at 30-40° C.

32. The method of any of claims 1-31, wherein (c) is performed at about 37° C.

33. The method of any of claims 1-32, wherein (c) is performed for less than or equal to 4 hours.

34. The method of any of claims 1-33, wherein the nucleic acid molecule comprises a 5-hydroxymethylcytosine (5hmC), the method further comprising incubating the nucleic acid molecule with a beta-glucosyltransferase (βGT) enzyme to glycosylate the 5hmC to 5-glyceryl-methylcytosine (5gmC) prior to (a).

35. The method of any of claims 1-34, wherein the method does not comprise bisulfite treatment.

36. The method of any of claims 1-35, wherein (c) is performed at a pH from 5.5 to 6.5.

37. A method for modifying a nucleic acid molecule comprising an abasic site, the method comprising incubating the nucleic acid molecule and a nucleobase derivative under conditions sufficient to attach the nucleobase derivative to the nucleic acid molecule at the abasic site.

38. The method of claim 37, wherein the nucleobase derivative comprises an azide moiety.

39. The method of claim 37 or 38, wherein the nucleobase derivative is a thymine derivative.

40. The method of claim 39, wherein the thymine derivative is a compound of formula I: 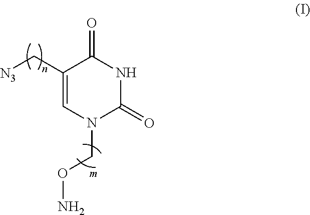
wherein n is an integer from 0 to 5 and m is an integer from 1 to 5.

41. The method of claim 40, wherein n is 3.

42. The method of claim 40, wherein m is 2.

43. The method of claim 40, wherein the thymine derivative is N₃-T:

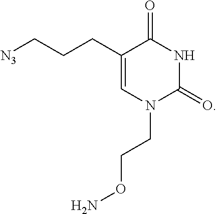 [SMILES: [N-]=[N+]=NCCCc1cn(CCON)c(=O)[nH]c1=O]

44. The method of claim 40, wherein the thymine derivative is a compound of formula (II): 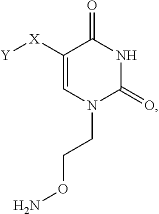
wherein X is a linker and Y is a click chemistry compatible reactive group selected from alkynes, azides, strained alkynes, dienes, dieneophiles, alkoxyamines, carbonyls, phosphines, hydrazides, thiols, alkenes, tetrazines, tetrazoles, isocyanates, isothiocyanates, and 1,3-nitrones.

45. The method of claim 44, wherein X is a C₁ to C₁₀ alkyl linker.

46. The method of claim 44, wherein X is an amide linker.

47. The method of claim 44, wherein X is an alkyl and aryl mixture linker.

48. The method of claim 44, wherein X is an alkyl and heterocycle mixture linker.

49. The method of claim 44, wherein Y is azide.

50. The method of claim 37 or 38, wherein the nucleobase derivative is an adenine derivative.

51. The method of any of claims 37-50, wherein the nucleic acid molecule is a deoxyribonucleic acid (DNA) molecule.

52. The method of any of claims 37-51, further comprising subjecting the nucleic acid molecule to a click chemistry reaction to attach a label to the nucleic acid molecule.

53. The method of claim 52, wherein the label is a dibenzocyclooctyne-modified biotin (DBCO-biotin).

54. The method of any of claims 50-53, wherein the method is performed at a pH from 5.5 to 6.5.

55. The method of any of claims 50-54, wherein the method is performed at 30-40° C.

56. The method of any of claims 50-55, wherein the method is performed at about 37° C.

57. The method of any of claims 50-56, wherein the method is performed for less than or equal to 4 hours.

58. A method for 5-methylcytosine (5mC) detection, the method comprising:
(a) incubating a nucleic acid molecule comprising a 5mC with a TET enzyme to oxidize 5mC to 5caC or 5fC;
(b) incubating the nucleic acid molecule with a TDG enzyme to excise the 5caC or 5fC and generate an abasic site;
(c) incubating the nucleic acid molecule with a thymine derivative comprising an azide moiety to attach the thymine derivative to the abasic site; and
(d) sequencing the nucleic acid molecule.

59. The method of claim 58, wherein the method does not comprise bisulfite treatment.

60. The method of claim 58 or 59, wherein the thymine derivative is N₃-thymine.

61. The method of any of claims 58-60, further comprising, prior to (d), incubating the nucleic acid molecule with a label comprising an alkyne moiety to attach the label to the thymine derivative.

62. A compound of formula (I): 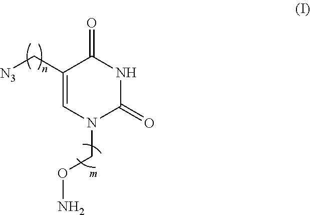
wherein n is an integer from 0 to 5 and m is an integer from 1 to 5.

63. The compound of claim 62, wherein n is 3.

64. The compound of claim 62, wherein m is 2.

65. The compound of any of claims 62-64, wherein the compound is further defined as:

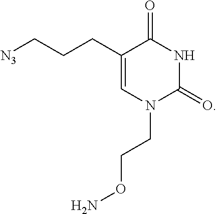 [SMILES: [N-]=[N+]=NCCCc1cn(CCON)c(=O)[nH]c1=O]

66. A compound having formula (II): 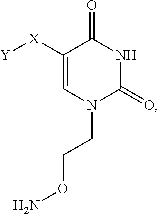
wherein X is a linker and Y is a click chemistry compatible reactive group selected from alkynes, azides, strained alkynes, dienes, dieneophiles, alkoxyamines, carbonyls, phosphines, hydrazides, thiols, alkenes, tetrazines, tetrazoles, isocyanates, isothiocyanates, and 1,3-nitrones.

67. The composition of claim 66, wherein X is a C₁ to C₁₀ alkyl linker.

68. The composition of claim 67, wherein X is CH₂.

69. The composition of claim 67, wherein X is CH₂CH₂.

70. The composition of claim 67, wherein X is CH₂CH₂CH₂.

71. The composition of claim 66, wherein X is a polyethylene glycol linker.

72. The method of claim 71, wherein X is an amide linker.

73. The method of claim 71, wherein X is an alkyl and aryl mixture linker.

74. The method of claim 71, wherein X is an alkyl and heterocycle mixture linker.

75. The composition of any of claims 66-74, wherein Y is azide.

76. A compound having formula (III): 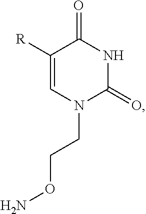
wherein R is H or alkyl.

77. The composition of claim 76, wherein R is methyl, ethyl, n-propyl, isopropyl, n-butyl, iso-butyl, or sec-butyl.

78. The composition of claim 76, wherein R is methyl.

79. The composition of claim 76, wherein the compound is further defined as: [chemical structure]
